(19) United States
(12) Patent Application Publication
(10) Pub. No.: US 2024/0255531 A1
(43) Pub. Date: Aug. 1, 2024

(54) MATURE ADRENOMEDULLIN FOR THERAPY STRATIFICATION OF CORTICOSTEROIDS IN CRITICALLY ILL PATIENTS

(71) Applicant: SPHINGOTEC GMBH, Hennigsdorf (DE)

(72) Inventors: Deborah BERGMANN, Berlin (DE); Florian UHLE, Heidelberg (DE)

(73) Assignee: SPHINGOTEC GMBH, Hennigsdorf (DE)

(21) Appl. No.: 18/289,612

(22) PCT Filed: May 6, 2022
(86) PCT No.: PCT/EP2022/062322

§ 371 Date: Nov. 6, 2023

(30) Foreign Application Priority Data
May 7, 2021 (EP) 21172844.9
Oct. 8, 2021 (EP) 21201767.7

(51) Int. Cl. G01N 33/74

(52) U.S. Cl. CPC G01N 33/743; G01N 2800/52

(57) ABSTRACT

A method of selecting critically ill patients for treatment with corticosteroids, by determining the level of ADM-NH₂ or fragment thereof in a sample of bodily fluid of the patient, comparing the level of ADM-NH₂ or fragment thereof to a pre-determined threshold or to a previously measured level of ADM-NH₂ or fragment thereof, and at a value above the threshold appointing a therapy with corticosteroids. A method for corticosteroid therapy guidance and/or stratification in critically ill patients by providing a sample of bodily fluid of the patient, and determining the level of ADM-NH₂ or fragment thereof in the sample, and comparing the level of ADM-NH₂ or fragment thereof to a pre-determined threshold or to a previously measured level of ADM-NH₂ or fragment thereof, wherein the level of ADM-NH₂ or fragments thereof is indicative of whether an initiation of a corticosteroid therapy is required.

### FIELD OF THE INVENTION

Subject matter of the present invention is a method of selection of critically ill patients for treatment with corticosteroids, which comprises determining the level of ADM-NH₂ or fragment thereof in a sample of bodily fluid of said patient, comparing said level of ADM-NH₂ or fragment thereof to a pre-determined threshold or to a previously measured level of ADM-NH₂ or fragment thereof, and at a value above said threshold appointing a therapy with corticosteroids, and at a value below said threshold renouncing a therapy with corticosteroids. Subject-matter of the present invention is also a method for corticosteroid therapy guidance and/or stratification in critically ill patients, the method comprising providing a sample of bodily fluid of said patient, and determining the level of ADM-NH₂ or fragment thereof in said sample, and comparing said level of ADM-NH₂ or fragment thereof to a pre-determined threshold or to a previously measured level of ADM-NH₂ or fragment thereof, wherein the level of ADM-NH₂ or fragments thereof is indicative of whether an initiation of a corticosteroid therapy is required.

Subject matter of the present invention is further a method of corticosteroid therapy guidance and/or corticosteroid therapy stratification of critically ill patients, the method comprising:
providing a sample of bodily fluid of said patient, and
determining the level of ADM-NH₂ or fragment thereof in said sample, and
comparing said level of ADM-NH₂ or fragment thereof to a pre-determined threshold or to a previously measured level of ADM-NH₂ or fragment thereof,
   wherein the level of ADM-NH₂ or fragments thereof is indicative of whether a corticosteroid therapy or an initiation of corticosteroid therapy is required.

### BACKGROUND

The peptide adrenomedullin (ADM) was described for the first time in Kitamura et al. (Kitamura et al. 1993. Biochemical and Biophysical Research Communications 192 (2): 553-560) as a novel hypotensive peptide comprising 52 amino acids, which had been isolated from a human pheochromocytoma. In the same year, cDNA coding for a precursor peptide comprising 185 amino acids and the complete amino acid sequence of this precursor peptide were also described. The precursor peptide, which comprises, inter alia, a signal sequence of 21 amino acids at the N-terminus, is referred to as “preproadrenomedullin” (pre-proADM). Pre-proADM comprises 185 amino acids (SEQ ID No.: 1). Pro-ADM is further processed into pro-ADM N-terminal 20 peptide (PAMP; SEQ ID No. 2), midregional pro-ADM (MR-proADM; SEQ ID No. 3), adrenotensin pro-ADM 153-185 (CT-pro ADM; SEQ ID No.: 6) and immature ADM, a C-terminally glycine-extended version of ADM (ADM-Gly; SEQ ID No. 5). This is converted into the mature bioactive form of ADM (bio-ADM; ADM-NH₂; SEQ ID No. 4), comprising 52 amino acids, by enzymatic amidation of its C-terminus.

The discovery and characterization of ADM in 1993 triggered intensive research activity, the results of which have been summarized in various review articles, in the context of the present description, reference being made in particular to the articles to be found in an issue of “Peptides” devoted to ADM in particular (Takahashi 2001. Peptides 22: 1691; Eto 2001. Peptides 22: 1693-1711). A further review is Hinson et al. 2000 (Hinson et al. 2000. Endocrine Reviews 21(2): 138-167). In the scientific investigations to date, it has been found, inter alia, that ADM may be regarded as a polyfunctional regulatory peptide. As mentioned above, it is released into the circulation in an inactive form extended by glycine (Kitamura et al. 1998. Biochem Biophys Res Commun 244(2): 551-555). There is also a binding protein (Pio et al. 2001. The Journal of Biological Chemistry 276(15): 12292-12300), which is specific for ADM and probably likewise modulates the effect of ADM. Those physiological effects of ADM as well as of PAMP, which are of primary importance in the investigations to date, were the effects influencing blood pressure.

Hence, ADM is an effective vasodilator, and thus it is possible to associate the hypotensive effect with the particular peptide segments in the C-terminal part of ADM. It has furthermore been found that the above-mentioned physiologically active peptide PAMP formed from pre-proADM likewise exhibits a hypotensive effect, even if it appears to have an action mechanism differing from that of ADM (in addition to the mentioned review articles above, Eto et al. 2001 and Hinson et al. 2000 see also Kuwasaki et al. 1997. FEBS Lett 414(1): 105-110; Kuwasaki et al. 1999. Ann. Clin. Biochem. 36: 622-628; Tsuruda et al. 2001 Life Sci. 69(2): 239-245 and EP-A2 0 622 458). It has furthermore been found, that the concentrations of ADM, which can be measured in the circulation and other biological liquids, are in a number of pathological states, significantly above the concentrations found in healthy control subjects. Thus, the ADM level in patients with congestive heart failure, myocardial infarction, kidney diseases, hypertensive disorders, diabetes mellitus, in the acute phase of shock and in sepsis and septic shock are significantly increased, although to different extents. The PAMP concentrations are also increased in some of said pathological states, but the plasma levels are lower relative to ADM (Eto 2001. Peptides 22: 1693-1711). It was reported that high concentrations of ADM are observed in sepsis, and the highest concentrations in septic shock (Eto 2001. Peptides 22: 1693-1711; Hirata et al. Journal of Clinical Endocrinology and Metabolism 81(4): 1449-1453; Ehlenz et al. 1997. Exp Clin Endocrinol Diabetes 105: 156-162; Tomoda et al. 2001. Peptides 22: 1783-1794; Ueda et al. 1999. Am. J. Respir. Crit. Care Med. 160: 132-136 and Wang et al. 2001. Peptides 22: 1835-1840). Moreover, plasma concentrations of ADM are elevated in patients with heart failure and correlate with disease severity (Hirayama et al. 1999. J Endocrinol 160: 297-303; Yu et al. 2001. Heart 86: 155-160). High plasma ADM is an independent negative prognostic indicator in these subjects (Poyner et al. 2002. Pharmacol Rey 54: 233-246).

### Indications

Sepsis is a multifaceted host response to an infecting pathogen that may be significantly amplified by endogenous factors. The original conceptualization of sepsis as infection with at least 2 of the 4 SIRS criteria focused solely on inflammatory excess. However, the validity of SIRS as a descriptor of sepsis pathobiology has been challenged. Sepsis is now recognized to involve early activation of both, pro- and anti-inflammatory responses, along with major modifications in non-immunologic pathways such as cardiovascular, neuronal, autonomic, hormonal, bioenergetic, metabolic, and coagulation. Today sepsis is defined, according to the Third International Consensus Definitions for Sepsis and Septic Shock (Sepsis-3), as life-threatening organ dysfunction caused by a dysregulated host response to infection (Singer et al. 2016. JAMA. 315 (8): 801-10). For clinical operationalization, organ dysfunction can be represented by an increase in the Sequential [Sepsis-related] Organ Failure Assessment (SOFA) score of 2 points or more, which is associated with an in-hospital mortality greater than 10%.

Septic shock is a potentially fatal medical condition that occurs when sepsis, which is organ injury or damage in response to infection, leads to dangerously low blood pressure and abnormalities in cellular metabolism. The Third International Consensus Definitions for Sepsis and Septic Shock (Sepsis-3) defines septic shock as a subset of sepsis in which particularly profound circulatory, cellular, and metabolic abnormalities are associated with a greater risk of mortality than with sepsis alone. Patients with septic shock can be clinically identified by a vasopressor requirement to maintain a mean arterial pressure of 65 mm Hg or greater and serum lactate level greater than 2 mmol/L (>18 mg/dL) in the absence of hypovolemia. This combination is associated with hospital mortality rates greater than 40% (Singer et al. 2016. JAMA. 315 (8): 801-10). The primary infection is most commonly caused by bacteria, but also may be by fungi, viruses or parasites. It may be located in any part of the body, but most commonly in the lungs, brain, urinary tract, skin or abdominal organs. It can cause multiple organ dysfunction syndrome (formerly known as multiple organ failure) and death. Frequently, people with septic shock are cared for in intensive care units. It most commonly affects children, immunocompromised individuals, and the elderly, as their immune systems cannot deal with infection as effectively as those of healthy adults. The mortality rate from septic shock is approximately 25-50%.

The Surviving Sepsis Campaign International Guidelines for the management of sepsis and septic shock 2016 recommends the use of IV hydrocortisone in septic shock patients where hemodynamic stability cannot be archived by fluid resuscitation and vasopressor therapy (refractory septic shock). The guidelines suggest against the use of IV hydrocortisone to treat patients with septic shock in case adequate fluid resuscitation and vasopressor administration allow hemodynamic stabilization (Rhodes et al. 2017. Intensive Care Med 43(3): 304-377).

Large clinical trials testing hydrocortisone therapy in septic shock have produced conflicting results. Subgroups may benefit of hydrocortisone treatment depending on their individual immune response. Though prospective, randomized, controlled multicenter trials have consistently reported faster shock resolution (Annane et al. 2009. JAMA. (2009) 301:2362-75; Venkatesh et al. 2018. N Engl J Med. 378:797-808), the utility of “low-dose” (200 mg/day) hydrocortisone (HC) in patients with septic shock remains controversial. Whereas, two French studies reported outcome benefit from a combination of hydrocortisone plus oral fludrocortisone (Annane et al. 2002. JAMA 288:862-71; Annane et al. 2018. N Engl J Med 378: 809-18), the pan-European CORTICUS trial and the 5-country ADRENAL trial found no survival effect from hydrocortisone alone (Venkatesh et al. 2018. N Engl J Med. 378:797-808; Sprung et al. 2008. N Engl J Med 358:111-24). It is increasingly recognized that patients presenting in septic shock are hyper-inflamed yet at the same time immunosuppressed (Hotchkiss et al. 2013. Nat Rev Immunol 13: 862-746; Kaufmann et al. 2018. Nat Rev Drug Discov 17:35-56). Corticosteroids are traditionally considered to induce immune suppression via the glucocorticoid receptor (GR) and its repressive effect on pro-inflammatory transcription factors such as AP-1 and NFkB (Baschant et al. 2013. Mol Cell Endocrinol 380: 108-18). Thus, patients in an overall state of immunosuppression may be potentially compromised by administration of an immunosuppressive drug. This argument is, however, complicated by increasing evidence implicating corticosteroids and GRs in immune-reconstitutive processes. This immune-activating role of corticosteroids has been described as a response to acute stress enhancing the peripheral immune response, whereas chronic corticosteroid exposure leads to immune suppression (Cruz-Topete et al. 2015. Neuroimmunomodulation 22: 20-32). These diverging effects of corticosteroids in respect to timing and benefits over side effects support the need for biomarkers to guide their application.

Beneficial effects of hydrocortisone therapy in patients with severe pneumonia were observed in a double-blind trial, investigating the effects of early administration of low dose hydrocortisone in patients at risk for sepsis. The findings of this study would support the potential of early treatment with hydrocortisone to prevent the development of life-threatening sepsis-related complications such as septic shock (Confalonieri et al. 2005. Am J Respir Crit Care Med Vol 171: 242-248), but a larger randomized, double-blind clinical trial to confirm these findings (the HYPRESS study) found that the preventive administration of hydrocortisone doesn't reduces the risk of a septic shock within 14 days. Other secondary endpoints, as mortality, were as well not improved by the treatment (Keh et al. 2016. JAMA 316(17): 1775-1785).

Side effects of hydrocortisone are described in literature. Higher numbers of secondary infections are reported in the CORTICUS study, whereas the ADRENAL and HYPRESS study could not confirm these findings.

Recently, it was shown that the ratio of serum interferon gamma (IFy) and interleukin 10 (IL-10) was a promising biomarker to guide the treatment decision for or against hydrocortisone (Koenig et al. 2021. Front Immunol 12:607217).

Low-dose corticosteroids were shown to significantly decrease pro-ADM levels in early stages of sepsis in a mouse model (Prasteyo et al. 2014. MKB 46(2): 68-72). In contrast, MR-proADM concentrations did not change significantly after fludrocortisone or dexamethasone treatment of patients with hypertension (Vogt et al. 2012. Clinical and Experimental Hypertension 34(8); 582-587).

WO2019077082 describes a method for monitoring a therapy in a subject, wherein the subject is under treatment with a binder against adrenomedullin by determining the level of a fragment of pre-pro-Adrenomedullin selected from the group comprising Midregional Proadrenomedullin (MR-proADM), C-terminal Proadrenomedullin (CT-proADM) and/or Proadrenomedullin N-terminal 20 peptide (PAMP) or fragments thereof (but not the mature ADM) in a bodily fluid obtained from said subject; and correlating said level of the fragment of pre-pro-Adrenomedullin with the requirement for adapting therapeutic measures of said patient, where said therapeutic measures is selected from the group comprising hydrocortisone.

Corona viruses are widespread in humans and several other vertebrates and cause respiratory, enteric, hepatic, and neuro logic diseases. Notably, the severe acute respiratory syndrome coronavirus (SARS-COV) in 2003 and Middle East respiratory syndrome coronavirus (MERS-CoV) in 2012 have caused human epidemics. Comparison with the SARS-COV shows several significant differences and similarities. Both MERS CoV and SARS-COV have much higher case fatality rates (40% and 10%, respectively) (de Wit et al. 2016. SARS and MERS: recent insights into emerging coronaviruses. Nat Rev Microbiol 14(8):523-34; Zhou et al. 2020. A pneumonia outbreak associated with a new coronavirus of probable bat origin. Nature 579(7798):270-273). Though the current SARS COV-2 shares 79% of its genome with SARS-CoV, it appears to be much more transmissible. The disease caused by SARS-COV-2 is called corona-virus-disease 2019 (COVID-19).

There is no specific, effective treatment or cure for COVID-19 (Siemieniuk et al. 2020. BMJ. 370: m2980). Thus, the cornerstone of management of COVID-19 is supportive care, which includes treatment to relieve symptoms, fluid therapy, oxygen support and prone positioning as needed, and medications or devices to support other affected vital organs, e.g., extracorporeal membrane oxygenation (ECMO). The role of steroids in this disease has been debated as well. The use of glucocorticoids in COVID-19 for treatment is discussed controversial (for review see Golamari et al. 2021. J Com Hosp Int Med Perspect 11(2): 187-193). One study reported that methylprednisolone may be beneficial, leading to decreased risk of death in patients with ARDS (Wu et al. 2020. JAMA Intern Med. 2020; 180(7):934-943). Some studies have shown a beneficial effect with low dose prednisone in cancer patients with COVID-19 (Russell et al. 2020. ecancer 14:1023) A metanalysis of one randomized-controlled trial and 22 cohort studies showed that glucocorticoid therapy reduced the duration of fever but did not affect the mortality, duration of hospitalization or lung inflammation absorption (Lu et al. 2020. Ann Transl Med 8(10):627).

A recently published open-labelled trial which studied dexamethasone vs usual care showed 28-day mortality benefit in those patients receiving invasive mechanical ventilation or oxygen with dexamethasone (Recovery trial). However, the positive results only applied to patients receiving respiratory oxygen support (Horby et al. 2020. NEJM DOI: 10.1056/NEJMoa 2021436).

Taken together, it is an unmet medical need to stratify critically ill patients (suffering from e.g., sepsis, septic shock or COVID-19) for treatment with corticosteroids, in particular glucocorticoids like hydrocortisone or dexamethasone. It is a specific unmet medical need to stratify patients with sepsis for treatment with hydrocortisone for the prevention of septic shock. It is another specific unmet medical need to stratify patients with COVID-19 for treatment with dexamethasone.

It was the surprising finding of the present invention that in critically ill patients when the level of ADM-NH₂ or fragments thereof in a sample of bodily fluid of said patient is below a pre-determined threshold, the administration of corticosteroids is contraindicated in said patient. Acute respiratory distress syndrome (ARDS) is a type of respiratory failure characterized by rapid onset of widespread inflammation in the lungs. Symptoms include shortness of breath, rapid breathing, and bluish skin coloration. ARDS is associated with a high mortality rate (35-45%) (Bellani et al. 2016. JAMA 315(8):788-800). For those who survive, a decreased quality of life is common. Causes may include sepsis, pancreatitis, trauma, pneumonia, and aspiration. The underlying mechanism involves diffuse injury to cells which form the barrier of the microscopic air sacs of the lungs, surfactant dysfunction, activation of the immune system, and dysfunction of the body's regulation of blood clotting. In effect, ARDS impairs the lungs' ability to exchange oxygen and carbon dioxide. Diagnosis is based on a PaO₂/FiO₂ ratio (ratio of partial pressure arterial oxygen and fraction of inspired oxygen) of less than 300 mm Hg despite a positive end-expiratory pressure (PEEP) of more than 5 cm H₂O. The primary treatment involves mechanical ventilation together with treatments directed at the underlying cause. Ventilation strategies include using low volumes and low pressures. If oxygenation remains insufficient, lung recruitment maneuvers and neuromuscular blockers may be used. If this is insufficient, extracorporeal membrane oxygenation (ECMO) may be an option.

Nine trials have investigated prolonged glucocorticoid treatment in ARDS (for review see Annane et al. 2017. Intensive Care Med 43:1751-1763). One of these trials was in patients with ARDS due to community-acquired pneumonia (Confalonieri et al. 2005. Am J Respir Crit Care Med 171(3):242-24859) and another was a subgroup analysis of the initial corticosteroid trial in septic shock (Annane et al. 2006. Crit Care Med 34(1):22-30). These trials consistently found that glucocorticoid treatment was associated with a significant reduction in markers of systemic inflammation (inflammatory cytokines and/or C-reactive protein levels), reduction in the duration of mechanical ventilation by approximately 7 days, and probable reduction in hospital mortality by approximately 7 and 11% in patients with mild and severe ARDS, respectively. However, their use in ARDS is still controversial, and the current society of critical care medicine guidelines have conditional recommendations for the use of glucocorticoids in patients with moderate-to-severe ARDS (Annane et al. 2017. Intensive Care Med 43:1751-1763). The guidelines conditionally recommend the use of methylprednisolone in early ARDS (up to day 7 of onset) at a dose of 1 mg/kg/d; for late persistent ARDS (after day 6 of onset), the guidelines recommend a dose of 2 mg/kg/d followed by gradual tapering. The timing of initiation of glucocorticoid therapy for ARDS is another issue of interest. Glucocorticoid treatment initiation within 1 week of ARDS onset provided a significant survival benefit, while treatment initiation at >1 week after ARDS diagnosis did not. This indicates that the use of glucocorticoids after the stage of irreversible lung injury may not offer much of a benefit. Therefore, a tool to distinguish between patients with ARDS especially in the late stage, who will benefit from glucocorticoid therapy is needed. Thus, the present invention enables to select patients with late persistent ARDS for treatment/therapy with corticosteroids.

Data showing a clinically significant mortality benefit of corticosteroids in the treatment of patients with severe CAP are limited. Some meta-analyses have found a reduced risk of death with corticosteroid use in such patients (Horita et al. 2015. Sci Rep 5:14061; Siemienieuk et al. 2015. Ann Intern Med 163(7):519-528; Jiang et al. 2019. Medicine (Baltimore) 98(26):e16239) but others have not (Briel et al. 2018. Clin Infect Dis 66(3): 346-354; Chen et al. 2015. World J Emerg Med 6(3): 172-178) and the studies included in the meta-analyses varied in their quality and their definitions of severe CAP. Moreover, there is no evidence that corticosteroids reduce mortality rates or other adverse clinical outcomes in patients with mild to moderate CAP. The new ATS/IDSA guidelines advise against adjunctive corticosteroid treatment of CAP or influenza pneumonia except in patients who have other indications for their use, such as asthma, COPD, or an autoimmune disease (Metlay et al. 2019. Am J Respir Crit Care Med Vol 200, Iss 7, pp e45-e67). Therefore, it is an unmet medical need for corticoid therapy stratification in patients with CAP, especially in mild to moderate CAP. Thus, the present invention enables to select patients with mild to moderate CAP for treatment/therapy with corticosteroids.

Cardiac surgery with the use of cardiopulmonary bypass results in a systemic inflammatory response; corticosteroids have been widely used to mitigate the potential deleterious effects of this response. In cardiopulmonary bypass surgery (CPB), also known as the ‘heart-lung machine’, cannulae are placed in the patient's major blood vessels and blood is channeled out of the body, oxygen is added, carbon dioxide is removed and the blood is then pumped back to the body. This allows the heart to be stopped and emptied of blood, thus allowing the surgeon to operate in a bloodless field on a non-beating heart (Barry et al. 2015. Anesthesia and Analgesia 120(4):749-769). As a result, there is activation of white blood cells and platelets, as well as coagulation cascades (Tarnok et al. 2001. Shock 16 (Suppl 1): 24-32), with the endsignalling due to cytokines. Endothelial permeability increases and parenchymal damage by free radicals occurs (Fudulu et al. 2016. Oxidative Medicine and Cellular Longevity 2016: 1971452; Pesonen et al. 2016. Acta Anaesthesiologica Scandinavica 60(10): 1386-1394).

Fluid leaks out of the circulation and into the tissues, blood vessels vasodilate, hypovolaemia occurs and thus poor blood pressure results. Many of the complications of cardiac surgery, including multi-organ failure and death, result from these mechanisms (Huffmyer et al. 2015. Best Practice & Research Clinical Anaesthesiology 29(2): 163-175). Nevertheless, the impact of prophylactic corticosteroids on clinical outcomes following heart surgery, especially on children, remains unclear. There is no consensus about whether to give corticosteroids or not (Fudulu et al. 2018. World Journal for Pediatric and Congenital Heart Surgery 9(3): 289-293), or about the type of corticosteroids, dose regimen or when they may be beneficial, e.g. pre-operatively versus intra-operatively versus post-operatively. Therefore, it is an unmet medical need to stratify patients undergoing cardiopulmonary bypass surgery for corticosteroid therapy.

Cardiac arrest occurs in over 400,000 patients in the United States each year and the overall mortality for cardiac arrest remains dismal with a survival rate less than 10% (Go et al. 2014. Circulation 129(3): e28-292). In an attempt to improve survival and quality of life, international cardiac arrest guidelines emphasize not only the importance of optimizing intra-arrest treatment, but also the management of patients during the post-cardiac arrest period (Field et al. 2010. Circulation 122 (18 Suppl 3): S640-56; Nolan et al. 2010. Resuscitation. 81(10): 1219-1276). The post-cardiac arrest syndrome is characterized by a variety of pathophysiological features similar to septic shock states including a systemic inflammatory response and hemodynamic perturbations, which may include microcirculatory dysfunction and myocardial suppression (Adrie et al. 2002. Circulation 106 (5): 562-568; Nolan et al. 2008. Resuscitation 79(3): 350-379). However, the utility and potential efficacy of corticosteroid therapy in post-cardiac arrest patients with shock remains unknown and only a few studies exist with different results (Mentzelopoulos et al. 2013. JAMA. 310(3):270-279; Donnino et al. 2016. Critical Care 20: 82).

Bacterial meningitis is a severe infection of the meninges (the membrane lining of the brain and spinal cord) that is associated with high mortality and morbidity rates despite optimal antibiotic therapy and advances in critical care. It is caused by bacteria that usually spread from an ear or respiratory infection and is treated with antibiotics. Late sequelae such as cranial nerve impairment, especially hearing loss, occur in 5% to 40% of patients. In experimental animal studies, treatment with corticosteroids was shown to result in a reduction of the inflammatory response in the cerebrospinal fluid (CSF), reversal of brain oedema and improved outcome (Scheld et al. 1980. Journal of Clinical Investigation 66 (2): 243-253); Tauber et al. 1985. Journal of Infectious Diseases 51(3): 528-534). These pathophysiological insights prompted investigators to evaluate corticosteroids as an adjuvant therapy in acute bacterial meningitis. However, clinical trials that evaluated the effect of corticosteroids in patients bacterial meningitis showed conflicting results (for review see Brouwer et al. 2015. Cochrane Database of Systematic Reviews Issue 9. Art. No.: CD004405). Therefore, it is an unmet medical need for corticoid therapy stratification in patients with meningitis, especially bacterial meningitis, who will benefit from this therapy.

### DETAILED DESCRIPTION

Subject matter of the present invention is a method of selection of critically ill patients for treatment with corticosteroids, which comprises determining the level of ADM-NH₂ or fragment thereof in a sample of bodily fluid of said patient, comparing said level of ADM-NH₂ or fragment thereof to a pre-determined threshold or to a previously measured level of ADM-NH₂ or fragment thereof, and at a value above said threshold appointing a therapy with corticosteroids, and at a value below said threshold renouncing a therapy with corticosteroids.

Subject matter of the present invention is also a method for corticosteroid therapy guidance and/or corticosteroid therapy stratification in critically ill patients, the method comprising
a. Providing a sample of bodily fluid of said patient, and
b. Determining the level of ADM-NH₂ or fragment thereof in said sample
c. Comparing said level of ADM-NH₂ or fragment thereof to a pre-determined threshold or to a previously measured level of ADM-NH₂ or fragment thereof,
   wherein the level of ADM-NH₂ or fragments thereof is indicative of whether an initiation of a corticosteroid therapy is required.

One embodiment of the present invention relates to a method for corticosteroid therapy guidance and/or stratification in critically ill patients, wherein said critically ill patients are patients suffering from a disease selected from the group of severe infection, sepsis, septic shock, acute respiratory syndrome (ARDS), community acquired pneumonia (CAP), meningitis (e.g. bacterial or viral meningitis), corona virus infection disease (e.g. COVID-19), cardiopulmonary bypass surgery (CPB) and cardiac arrest.

In the context of the present invention, critically ill patient is a patient suffering from a disease selected from the group of severe infection, sepsis, septic shock, acute respiratory syndrome (ARDS), community acquired pneumonia (CAP), meningitis (e.g., bacterial or viral meningitis), corona virus infection disease (e.g., COVID-19), cardiopulmonary bypass surgery (CPB) and cardiac arrest.

In one embodiment of the present application a patient is a patient suffering from sepsis or septic shock.

In one embodiment of the present application, a patient is infected with a Corona virus, wherein the Corona Virus is selected from the group comprising Sars-COV-1, Sars-COV-2, MERS-COV, in particular Sars-COV-2.

In one embodiment of the present application, a patient is a patient suffering from an acute respiratory syndrome (ARDS). In a more specific embodiment, the patient with ARDS is a late stage of 7 or more days since onset of symptoms.

In one embodiment of the present application, a patient is a patient suffering from community acquired pneumonia (CAP). In a more specific embodiment, the patient has mild to moderate (non-severe) CAP. Severity of CAP is currently defined by the degree of physiological impairment, as classified by the IDSA/ATS 2007 criteria (Piffer et al. 2007. Breathe 4(2): 110-115).

In one embodiment of the present application, the patient is a patient suffering from meningitis, especially bacterial meningitis.

In the context of the present application, critically ill patients are not treated with any drugs, medicaments, antibodies, and/or other agents or therapy that leads to an increase of the level of ADM-NH₂ or fragments thereof in said patient, particularly antibodies, antibody fragments or non-Ig scaffolds specifically binding to ADM-NH₂.

In the context of the present application, critically ill patients may be treated in addition to corticosteroids with any drugs, medicaments or therapeutic agents that do not lead to an increase of the level of ADM-NH₂ or fragments thereof. Said additional drug, medication or therapeutic agent may be selected from the group comprising vasopressors, fluid therapy, antimicrobial therapy (including antibiotics and anti-viral agents), renal replacement therapy.

Another embodiment of the present invention relates to a method for corticosteroid therapy guidance and/or corticoid therapy stratification in critically ill patients, wherein the level of ADM-NH₂ or fragment thereof is determined by contacting said sample of bodily fluid with a capture binder that binds specifically to ADM-NH₂ or fragment thereof.

Another embodiment of the present invention relates to a method for corticosteroid therapy guidance and/or corticosteroid therapy stratification in critically ill patients, wherein the level of ADM-NH₂ or fragment thereof is determined by contacting said sample of bodily fluid with a capture binder that binds specifically to the C-terminal part of ADM-NH₂ and wherein said capture binder specifically needs the C-terminal amide of ADM-NH₂ for binding.

Another specific embodiment of the present invention relates to a method for corticosteroid therapy guidance and/or corticoid therapy stratification in critically ill patients, wherein said determination comprises the use of a capture-binder that binds specifically to ADM-NH₂ or fragment thereof wherein said capture-binder may be selected from the group of antibodies, antibody fragment or non-IgG scaffold.

Another preferred embodiment of the present invention relates to a method for corticosteroid therapy guidance and/or corticoid therapy stratification in critically ill patients, wherein the level of ADM-NH₂ is determined in a bodily fluid sample of said subject and wherein said determination comprises the use of a capture-binder that binds specifically to level of ADM-NH₂, wherein said capture-binder is immobilized on a surface.

Another embodiment of the present invention relates to a method for corticosteroid therapy guidance and/or corticoid therapy stratification in critically ill patients, wherein the level of ADM-NH₂ is determined by different methods, e.g., immunoassays, activity assays, mass spectrometric methods.

Another embodiment of the present invention relates to a method for corticosteroid therapy guidance and/or corticoid therapy stratification in critically ill patients, wherein the assay sensitivity of said assay for the detection of ADM-NH₂ is able to quantify ADM-NH₂ of healthy subjects and is <70 pg/ml, preferably <40 pg/ml and more preferably <10 pg/ml.

One embodiment of the present invention relates to a method for corticosteroid therapy guidance and/or corticoid therapy stratification in critically ill patients, wherein said patient is treated with corticosteroids selected from the group consisting of glucocorticoids or mineralcorticoids.

Another embodiment of the present invention relates to a method for corticosteroid therapy guidance and/or corticoid therapy stratification in critically ill patients, wherein said glucocorticoids may be selected from the group comprising cortisone, hydrocortisone, prednisone, prednisolone, methylprednisolone, dexamethasone or betamethasone.

Another specific embodiment of the present invention relates to a method for corticosteroid therapy guidance and/or corticoid therapy stratification in critically ill patients, wherein said mineralcorticoids may be selected from the group comprising fludrocortisone.

One embodiment of the present invention relates to a method for corticosteroid therapy guidance and/or corticoid therapy stratification in critically ill patients, wherein said Corona Virus is selected from the group comprising Sars-COV-1, Sars-COV-2, MERS-COV, in particular Sars-COV-2.

Another embodiment of the present invention relates to a method for corticosteroid therapy guidance and/or corticoid therapy stratification in critically ill patients, wherein the bodily is selected from the group comprising whole blood, serum and plasma.

Another preferred embodiment of the present invention relates to a method for corticosteroid therapy guidance and/or corticoid therapy stratification in critically ill patients, wherein the threshold level of ADM-NH₂ is between 20 and 150 pg/mL, more preferred between 30 and 100 pg/mL, even more preferred between 40 and 80 pg/mL, most preferred said threshold level is 70 pg/mL.

Another embodiment of the present invention relates to a method for corticosteroid therapy guidance and/or corticoid therapy stratification in critically ill patients, wherein further biomarkers may be measured in addition to ADM-NH₂ or fragments thereof.

Another embodiment of the present invention relates to a method for corticosteroid therapy guidance and/or corticoid therapy stratification in critically ill patients, wherein said further biomarkers are selected from the group comprising D-Dimer, procalcitonin (PCT), C-reactive protein (CRP), lactate, penKid, NT-proBNP, BNP, white blood cell count, lymphocyte count, neutrophil count, hemoglobin, platelet count, albumin, alanine transaminase, creatinine, blood urea, lactate dehydrogenase, creatinin kinase, cardiac troponin I, prothrombin time, serum ferritin, interleukin-6 (IL-6), IL-10, IL-2, IL-7, interferon gamma (IF-γ), tumor necrosis factor-α (TNF-α), granulocyte colony-stimulating factor (GCSF), IP-10, MCP-1, MIP-1a.

Another embodiment of the present application relates to a method of selection of patients with sepsis for treatment with hydrocortisone for the prevention of septic shock, which consists in determining the level of ADM-NH₂ or fragment thereof in a sample of bodily fluid of said patient, comparing said level of ADM-NH₂ or fragment thereof to a pre-determined threshold or to a previously measured level of ADM-NH₂ or fragment thereof, and at a value above said threshold appointing a therapy with hydrocortisone, and at a value below said threshold renouncing a therapy with hydrocortisone.

Another embodiment of the present application relates to a method for hydrocortisone therapy guidance and/or corticoid therapy stratification in patients with sepsis for the prevention of septic shock, the method comprising
a. Providing a sample of bodily fluid of said patient, and
b. Determining the level of ADM-NH₂ or fragment thereof in said sample, and
c. Comparing said level of ADM-NH₂ or fragment thereof to a pre-determined threshold or to a previously measured level of ADM-NH₂ or fragment thereof,
   wherein the level of ADM-NH₂ or fragments thereof is indicative of whether an initiation of a hydrocortisone therapy is required.

Another embodiment of the present application relates to a method of selection of patients with COVID-19 for treatment with dexamethasone, which consists in determining the level of ADM-NH₂ or fragment thereof in a sample of bodily fluid of said patient, comparing said level of ADM-NH₂ or fragment thereof to a pre-determined threshold or to a previously measured level of ADM-NH₂ or fragment thereof, and at a value above said threshold appointing a therapy with dexamethasone, and at a value below said threshold renouncing a therapy with dexamethasone.

Another embodiment of the present application relates to a method for dexamethasone therapy guidance and/or corticoid therapy stratification in patients with COVID-19, the method comprising
a. Providing a sample of bodily fluid of said patient, and
b. Determining the level of ADM-NH₂ or fragment thereof in said sample, and
c. Comparing said level of ADM-NH₂ or fragment thereof to a pre-determined threshold or to a previously measured level of ADM-NH₂ or fragment thereof,
   wherein the level of ADM-NH₂ or fragments thereof is indicative of whether an initiation of a dexamethasone therapy is required.

Said hydrocortisone may be applied as a continuous infusion at a dose of 1 to 10 mg/h to said patient. Said hydrocortisone may be applied as a continuous infusion at a dose of 10 mg/h as long as vasopressors are given to said patient in parallel. Said hydrocortisone may be applied as a continuous infusion at a dose of 5 mg/h after stopping vasopressors. Said hydrocortisone dose may be reduced step-by-step (reduction of 1 mg/h per day) to 1 mg/h.

Said hydrocortisone may be applied as an intravenous bolus of 50 mg, followed by a 24-hour continuous infusion of 200 mg on 5 days, 100 mg on days 6 and 7, 50 mg on days 8 and 9, and 25 mg on days 10 and 11.

Hydrocortisone may be combined with fludrocortisone. In a specific embodiment a 50 mg bolus infusion of hydrocortisone is applied every 6 hours supplemented by daily oral fludrocortisone (50 μg).

In another specific embodiment of the present application, said corticosteroid therapy is initiated when said level of ADM-NH₂ is above a threshold level between 20 and 150 pg/mL, more preferred between 30 and 100 pg/mL, even more preferred between 40 and 80 pg/mL, most preferred said threshold level is 70 pg/mL.

In another specific embodiment of the present application, said corticosteroid therapy is continued when said level of ADM-NH₂ is above a threshold level between 20 and 150 pg/mL, more preferred between 30 and 100 pg/mL, even more preferred between 40 and 80 pg/mL, most preferred said threshold level is 70 pg/mL.

In another specific embodiment of the present application, said corticosteroid therapy is terminated when said level of ADM-NH₂ is below a threshold level between 20 and 150 pg/mL, more preferred between 30 and 100 pg/mL, even more preferred between 40 and 80 pg/mL, most preferred said threshold level is 70 pg/mL.

In another embodiment of the present application, the level of ADM-NH₂ is determined in a bodily fluid sample of said subject and wherein said determination comprises the use of a capture-binder that binds specifically to ADM wherein said capture-binder is an antibody.

In another embodiment of the present application, the level of ADM-NH₂ is determined in a bodily fluid sample of said subject and wherein said determination comprises the use of a capture-binder that binds specifically to level of ADM-NH₂, wherein said capture-binder is immobilized on a surface.

In another embodiment of the present application, the patient is treated with corticosteroids, wherein said corticosteroids are selected from the group consisting of glucocorticoids or mineralcorticoids.

In another embodiment of the present application, glucocorticoids may be selected from the group comprising cortisone, hydrocortisone, prednisone, prednisolone, methylprednisolone, dexamethasone or betamethasone.

In one embodiment of the present application, the mineralcorticoids may be selected from the group comprising fludrocortisone.

A bodily fluid according to the present invention is in one particular embodiment a blood sample. A blood sample may be selected from the group comprising whole blood, serum and plasma. In a specific embodiment of the diagnostic method said sample is selected from the group comprising human citrate plasma, heparin plasma and EDTA plasma.

In one embodiment the assay sensitivity of said assay for the detection of ADM-NH₂ is able to quantify ADM-NH₂ of healthy subjects and is <70 pg/ml, preferably <40 pg/ml and more preferably <10 pg/ml.

Further biomarkers may be measured in addition to ADM-NH₂ or fragments thereof. Said further biomarkers may be selected from the group comprising D-Dimer, procalcitonin (PCT), C-reactive protein (CRP), lactate, penKid, NT-proBNP, BNP, white blood cell count, lymphocyte count, neutrophil count, hemoglobin, platelet count, albumin, alanine transaminase, creatinine, blood urea, lactate dehydrogenase, creatinin kinase, cardiac troponin I, prothrombin time, serum ferritin, interleukin-6 (IL-6), IL-10, IL-2, IL-7, interferon gamma (IF-γ), tumor necrosis factor-α (TNF-α), granulocyte colony-stimulating factor (GCSF), IP-10, MCP-1, MIP-1α.

Subject matter of the present invention is also a method of selection of critically ill patients for treatment with corticosteroids, which comprises providing a sample of bodily fluid of said patient, determining the level of ADM-NH₂ or fragment thereof in said sample, comparing said level of ADM-NH₂ or fragment thereof to a pre-determined threshold or to a previously measured level of ADM-NH₂ or fragment thereof, and at a value above said threshold appointing a therapy with corticosteroids, and at a value below said threshold renouncing a therapy with corticosteroids.

One embodiment of the present invention relates to a method of selection of critically ill patients for treatment with corticosteroids, further comprising a step of corticosteroid therapy guidance and/or corticosteroid therapy stratification in critically ill patients, the method comprising
a. Providing a sample of bodily fluid of said patient, and
b. Determining the level of ADM-NH₂ or fragment thereof in said sample, and
C. Comparing said level of ADM-NH₂ or fragment thereof to a pre-determined threshold or to a previously measured level of ADM-NH₂ or fragment thereof,
wherein the level of ADM-NH₂ or fragments thereof is indicative of whether an initiation of a corticosteroid therapy is required.

Another embodiment of the present invention relates to a method of selection of critically ill patients for treatment with corticosteroids and of corticosteroid therapy guidance and/or corticosteroid therapy stratification in critically ill patients, wherein said critically ill patients are patients suffering from a disease selected from the group of severe infection, sepsis, septic shock, acute respiratory syndrome (ARDS), community acquired pneumonia (CAP), meningitis (e.g., bacterial or viral meningitis), corona virus infection disease (e.g., COVID-19), cardiopulmonary bypass surgery (CPB) and cardiac arrest.

Another embodiment of the present invention relates to method of selection of critically ill patients for treatment with corticosteroids and of corticosteroid therapy guidance and/or corticosteroid therapy stratification in critically ill patients, wherein the level of ADM-NH₂ or fragment thereof is determined by contacting said sample of bodily fluid with a capture binder that binds specifically to pro-Adrenomedullin or fragment thereof.

Another preferred embodiment of the present invention relates to a method of selection of critically ill patients for treatment with corticosteroids and of corticosteroid therapy guidance and/or corticosteroid therapy stratification in critically ill patients, wherein said determination comprises the use of a capture-binder that binds specifically to ADM-NH₂ or fragment thereof wherein said capture-binder may be selected from the group of antibodies, antibody fragment or non-IgG scaffold.

Another specific embodiment of the present invention relates to a method of selection of critically ill patients for treatment with corticosteroids and of corticosteroid therapy guidance and/or corticosteroid therapy stratification in critically ill patients, wherein the level of ADM-NH₂ is determined in a bodily fluid sample of said subject and wherein said determination comprises the use of a capture-binder that binds specifically to level of ADM-NH₂, wherein said capture-binder is immobilized on a surface.

Another embodiment of the present invention relates to a method of selection of critically ill patients for treatment with corticosteroids and of corticosteroid therapy guidance and/or corticosteroid therapy stratification in critically ill patients, wherein the level of ADM-NH₂ is determined by different methods, e.g. immunoassays, activity assays, mass spectrometric methods.

Another preferred embodiment of the present invention relates to a method of selection of critically ill patients for treatment with corticosteroids and of corticosteroid therapy guidance and/or corticosteroid therapy stratification in critically ill patients, wherein the assay sensitivity of said assay for the detection of ADM-NH₂ is able to quantify ADM-NH₂ of healthy subjects and is <70 pg/ml, preferably <40 pg/ml and more preferably <10 pg/ml.

Another embodiment of the present invention relates to a method of selection of critically ill patients for treatment with corticosteroids and of selecting corticosteroid therapy guidance and/or corticosteroid therapy stratification in critically ill patients, wherein said patient is treated with corticosteroids selected from the group consisting of glucocorticoids or mineralcorticoids.

Another specific embodiment of the present invention relates to a method of selection of critically ill patients for treatment with corticosteroids and of corticosteroid therapy guidance and/or corticosteroid therapy stratification in critically ill patients, wherein said glucocorticoids may be selected from the group comprising cortisone, hydrocortisone, prednisone, prednisolone, methylprednisolone, dexamethasone or betamethasone.

One embodiment of the present invention relates to a method of selection of critically ill patients for treatment with corticosteroids and of corticosteroid therapy guidance and/or corticosteroid therapy stratification in critically ill patients, wherein said mineralcorticoids may be selected from the group comprising fludrocortisone.

Another embodiment of the present invention relates to a method of selection of critically ill patients for treatment with corticosteroids and of corticosteroid therapy guidance and/or corticosteroid therapy stratification in critically ill patients, wherein said Corona Virus is selected from the group comprising Sars-COV-1, Sars-COV-2, MERS-COV, in particular Sars-CoV-2.

Another preferred embodiment of the present invention relates to a method of selection of critically ill patients for treatment with corticosteroids and of corticosteroid therapy guidance and/or corticosteroid therapy stratification in critically ill patients, wherein the bodily is selected from the group comprising whole blood, serum and plasma.

Another specific embodiment of the present invention relates to a method of selection of critically ill patients for treatment with corticosteroids and of corticosteroid therapy guidance and/or corticosteroid therapy stratification in critically ill patients, wherein the threshold level of ADM-NH₂ is between 20 and 150 pg/mL, more preferred between 30 and 100 pg/mL, even more preferred between 40 and 80 pg/mL, most preferred said threshold level is 70 pg/mL.

Another embodiment of the present invention relates to a method of selection of critically ill patients for treatment with corticosteroids and of corticosteroid therapy guidance and/or corticosteroid therapy stratification in critically ill patients, wherein further biomarkers may be measured in addition to ADM-NH₂ or fragments thereof.

Another preferred embodiment of the present invention relates to a method of selection of critically ill patients for treatment with corticosteroids and of corticosteroid therapy guidance and/or corticosteroid therapy stratification in critically ill patients, wherein said further biomarkers are selected from the group comprising D-Dimer, procalcitonin (PCT), C-reactive protein (CRP), lactate, penKid, NT-proBNP, BNP, white blood cell count, lymphocyte count, neutrophil count, hemoglobin, platelet count, albumin, alanine transaminase, creatinine, blood urea, lactate dehydrogenase, creatinin kinase, cardiac troponin I, prothrombin time, serum ferritin, interleukin-6 (IL-6), IL-10, IL-2, IL-7, interferon gamma (IF-γ), tumor necrosis factor-α (TNF-α), granulocyte colony-stimulating factor (GCSF), IP-10, MCP-1, MIP-1α.

Subject-matter of the present application are also corticosteroids for use in the treatment of critically ill patients, wherein said patients are characterized in that the level of ADM-NH₂ or fragment thereof in a sample of bodily fluid of said patient is at a value above a pre-determined threshold or to a previously measured level of ADM-NH₂ or fragment thereof, when said level of ADM-NH₂ or fragment thereof is compared to said pre-determined threshold or to a previously measured level of ADM-NH₂ or fragment thereof, wherein said critically ill patients are patients suffering from a disease selected from the group of severe infection, sepsis, septic shock, acute respiratory syndrome (ARDS), community acquired pneumonia (CAP), meningitis (e.g., bacterial or viral meningitis), corona virus infection disease (e.g., COVID-19), cardiopulmonary bypass surgery (CPB) and cardiac arrest.

Another embodiment of the present invention relates to corticosteroids for use in the treatment of critically ill patients, wherein said patients are characterized in that the level of ADM-NH₂ or fragment thereof has been determined in a sample of bodily fluid of said patient, wherein said level of ADM-NH₂ or fragment thereof has been compared to a pre-determined threshold or to a previously measured level of ADM-NH₂ or fragment thereof, and at a value above said threshold a therapy with corticosteroids is appointed, wherein said critically ill patients are patients suffering from a disease selected from the group of severe infection, sepsis, septic shock, acute respiratory syndrome (ARDS), community acquired pneumonia (CAP), meningitis (e.g., bacterial or viral meningitis), corona virus infection disease (e.g., COVID-19), cardiopulmonary bypass surgery (CPB) and cardiac arrest.

Another preferred embodiment of the present invention relates to corticosteroids for use in the treatment of critically ill patients, wherein said patient is treated with corticosteroids selected from the group consisting of glucocorticoids or mineralcorticoids.

Another embodiment of the present invention relates to corticosteroids for use in the treatment of critically ill patients, wherein said glucocorticoids may be selected from the group comprising cortisone, hydrocortisone, prednisone, prednisolone, methylprednisolone, dexamethasone or betamethasone.

One embodiment of the present invention relates to corticosteroids for use in the treatment of critically ill patients, wherein said glucocorticoids may be selected from the group comprising cortisone, hydrocortisone, prednisone, prednisolone, methylprednisolone, dexamethasone or betamethasone.

Another embodiment of the present invention relates to corticosteroids for use in the treatment of critically ill patients, wherein said Corona Virus is selected from the group comprising Sars-CoV-1, Sars-COV-2, MERS-COV, in particular Sars-COV-2.

Another preferred embodiment of the present invention relates to corticosteroids for use in the treatment of critically ill patients, wherein the bodily is selected from the group comprising whole blood, serum and plasma.

Another embodiment of the present invention relates to corticosteroids for use in the treatment of critically ill patients, wherein the level of ADM-NH₂ or fragment thereof is determined by contacting said sample of bodily fluid with a capture binder that binds specifically to ADM-NH₂ or fragment thereof.

Another embodiment of the present invention relates to corticosteroids for use in the treatment of critically ill patients, wherein said determination comprises the use of a capture-binder that binds specifically to ADM-NH₂ or fragment thereof wherein said capture-binder may be selected from the group of antibodies, antibody fragment or non-IgG scaffold.

Another embodiment of the present invention relates to corticosteroids for use in the treatment of critically ill patients, wherein the level of ADM-NH₂ is determined in a bodily fluid sample of said subject and wherein said determination comprises the use of a capture-binder that binds specifically to level of ADM-NH₂, wherein said capture-binder is immobilized on a surface.

Another embodiment of the present invention relates to corticosteroids for use in the treatment of critically ill patients, wherein the level of ADM is determined by different methods, e.g., immunoassays, activity assays, mass spectrometric methods.

Another embodiment of the present invention relates to corticosteroids for use in the treatment of critically ill patients, wherein the assay sensitivity of said assay for the detection of ADM-NH₂ is able to quantify ADM-NH₂ of healthy subjects and is <70 pg/ml, preferably <40 pg/ml and more preferably <10 pg/ml.

### Definitions

The severity of a disease is defined as the extent of organ system derangement or physiologic decompensation for a patient. The severity may be classified into different stages using for example scoring systems.

As used herein, organ dysfunction denotes a condition or a state of health where an organ does not perform its expected function. “Organ failure” denotes an organ dysfunction to such a degree that normal homeostasis cannot be maintained without external clinical intervention.

Said organ failure may pertain an organ selected from the group comprising kidney, liver, heart, lung, nervous system. By contrast, organ function represents the expected function of the respective organ within physiologic ranges. The person skilled in the art is aware of the respective function of an organ during medical examination.

Organ dysfunction may be defined by the sequential organ failure assessment score (SOFA-Score) or the components thereof. The SOFA score, previously known as the sepsis-related organ failure assessment score (Singer et al. 2016. JAMA 315(8):801-10) is used to track a person's status during the stay in an intensive care unit (ICU) to determine the extent of a person's organ function or rate of failure. The score is based on six different scores, one each for the respiratory, cardiovascular, hepatic, coagulation, renal and neurological systems each scored from 0 to 4 with an increasing score reflecting worsening organ dysfunction. The criteria for assessment of the SOFA score are described for example in Lamden et al. (for review see Lambden et al. 2019. Critical Care 23:374). SOFA score may traditionally be calculated on admission to ICU and at each 24-h period that follows. In particular, said organ dysfunction is selected from the group comprising renal decline, cardiac dysfunction, liver dysfunction or respiratory tract dysfunction.

The quick SOFA Score (quickSOFA or qSOFA) was introduced by the Sepsis-3 group in February 2016 as a simplified version of the SOFA Score as an initial way to identify patients at high risk for poor outcome with an infection (Angus et al. 2016. Critical Care Medicine. 44 (3): e113-e121). The qSOFA simplifies the SOFA score drastically by only including its 3 clinical criteria and by including “any altered mentation” instead of requiring a GCS<15. qSOFA can easily and quickly be repeated serially on patients. The score ranges from 0 to 3 points. One point is given for: low blood pressure (SBP≤100 mmHg), high respiratory rate ((≥22 breaths/min) and altered mentation (GCS≤15). The presence of 2 or more qSOFA points near the onset of infection was associated with a greater risk of death or prolonged intensive care unit stay. These are outcomes that are more common in infected patients who may be septic than those with uncomplicated infection. Based upon these findings, the Third International Consensus Definitions for Sepsis recommends qSOFA as a simple prompt to identify infected patients outside the ICU who are likely to be septic (Seymour et al. 2016. JAMA 315(8):762-774).

A life-threatening deterioration is defined as an acute condition of a patient associated with a high risk of death that involves vital organ system failure including central nervous system failure, renal failure, hepatic failure, metabolic failure or respiratory failure.

An adverse event is defined as death, organ dysfunction or shock.

Said clinical parameter or clinical scores are selected from the group comprising history of hypotension, vasopressor requirement, intubation, mechanical ventilation, Horovitz index, SOFA score, quick SOFA score.

The term “therapy stratification” in particular relates to grouping, selecting or classifying patients into different groups, such as therapy groups that receive or do not receive therapeutic measures depending on their classification.

The stratified patient groups may include patients that require an initiation of treatment and patients that do not require initiation of treatment.

As used herein, the term “therapy guidance” refers to application of certain therapies or medical interventions based on the value of one or more biomarkers and/or clinical parameter and/or clinical scores as well as to the monitoring of a therapy including adjustment of treatment with corticosteroids of said patients, for example by obtaining feedback on the efficacy of the therapy.

In one embodiment of the present invention said determination of ADM-NH₂ or fragments thereof is performed more than once in one critically ill patient.

In another embodiment of the present invention said monitoring is performed in order to evaluate the response of said critically ill patient to the treatment with corticosteroids.

Moreover, said patients may be stratified into one of the following groups:
(i) responder to said corticosteroid treatment of said disease showing a favourable effect after having received said treatment,
(ii) non-responder to said corticosteroid treatment of said disease showing no effect (neither a favourable nor an unfavourable effect) after having received said corticosteroid treatment,
(iii) patients showing an unfavourable effect after having received said corticosteroid treatment.

The data in Example 6 clearly demonstrate that corticosteroid (especially hydrocortisone) therapy stratification in sepsis patients using ADM-NH₂ may prevent the development of septic shock and shows that patients may benefit with a shortened time of hospital stay and less additional treatment.

Another particular advantage of the present invention is that the method can discriminate patients who are more likely to benefit from said therapy from patients who are not likely to benefit from said therapy.

Said benefit from corticoisteroid therapy may be for example the resolving of symptoms of the disease (pathophysiological symptoms, biomarker values etc.), the weaning of other life-supporting therapies or a positive outcome of the patient (e.g., survival).

In a preferred embodiment, the treatment is initiated or changed immediately upon provision of the result of the sample analysis indicating the level of ADM-NH₂ in the sample. In further embodiments, the treatment may be initiated within 12, preferably 6, 4, 2, 1, 0.5, 0.25 hours or immediately after receiving the result of the sample analysis.

Corticosteroids are steroid hormones that are either produced physiologically by vertebrates (natural corticosteroids) or are manufactured (synthetic corticosteroids). Endogenous corticosteroids are synthesized in the adrenal cortex and secreted into the blood to regulate a wide spectrum of physiological systems. All steroid hormones are synthesized from cholesterol and, in humans, the major secretions of the adrenal cortex are cortisol (member of the glucocorticoid family) and aldosterone (a member of the mineralocorticoid family) (for review see: Williams 2018. Respir Care 63(6):655-670). Glucocorticoids regulate lipid, glucose and protein metabolism, exert anti-inflammatory/immunosuppressive actions, and vasoconstrictive effects, whereas mineralocorticoids are the main regulators of electrolyte and water balance. Except for fludrocortisone and desoxycorticosterone acetate, the majority of synthetic corticosteroids mimics the actions of endogenous glucocorticoids (see table 1). Their clinical potency, indeed, is much higher than cortisol and they do not display mineralocorticoid effects. Corticosteroids systemically used are classified according to potency, mineralocorticoid effects, and duration of hypothalamic-pituitary-adrenal axis suppression. Potency is expressed relative to hydrocortisone and is useful in determining comparable doses. Mineralocorticoid activity is also described relative to hydrocortisone, and structural modifications to the steroid molecule are designed to increase potency as well as to minimize mineralocorticoid effects when these agents are used in pharmacologic doses to prevent or treat allergic, inflammatory, or immune responses. These agents are classified as short, medium, or long acting based on the duration of hypothalamic-pituitary-adrenal axis suppression.

TABLE 1
Corticosteroid comparison chart (adapted from Samuel
et al. 2017. J Neurocrit Care 10(2): 53-59)

| | Equivalent | | | |
|---|---|---|---|---|
| | glucocorticoid | Potency relat. to hydrocortisone | | Half-life duration |
| | dose (mg) | Anti-inflammatory | Mineralcorticoid | of action (hours) |
| Glucocorticoids | | | | |
| Short acting | | | | |
| Hydrocortisone | 20 | 1 | 1 | 8-12 |
| Cortisone acetate | 25 | 0.8 | 0.8 | 8-12 |
| Intermediate acting | | | | |
| Prednisone | 5 | 4 | 0.8 | 12-36 |
| Prednisolone | 5 | 4 | 0.8 | 12-36 |
| Methylprednisolone | 4 | 5 | 0.5 | 12-36 |
| Long acting | | | | |
| Dexamethasone | 0.75 | 30 | 0 | 36-54 |
| Mineralcorticoid | | | | |
| Fludrocortisone | 0 | 15 | 150 | 24-36 |

Said therapy or intervention are corticosteroids selected from the group consisting of glucocorticoids or mineralcorticoids. Glucocorticoids may be selected from the group comprising cortisone, hydrocortisone, prednisone, prednisolone, methylprednisolone, dexamethasone or betamethasone. Mineralcorticoids may be selected from the group comprising fludrocortisone.

Said corticosteroids may be applied to the patient as intravenous bolus or continuous infusion or may be administered orally.

The term “patient” as used herein refers to a living human or non-human organism that is receiving medical care or that should receive medical care due to a disease. This includes persons with no defined illness who are being investigated for signs of pathology. Thus, the methods and assays described herein are applicable to both, human and veterinary disease.

The term “critically ill patients” refers to patients suffering from an acute disease or acute condition. Said critically ill patient may be selected from the group comprising severe infection, sepsis, septic shock, acute respiratory syndrome (ARDS), community acquired pneumonia (CAP), meningitis (e.g., bacterial or viral meningitis), corona virus infection disease (e.g., COVID-19), cardiopulmonary bypass surgery (CPB) and cardiac arrest.

The patient suffering from such an acute disease or condition may have a co-morbidity like a chronic disease (e.g., cancer). The treatment according to the present invention aims especially the treatment of the acute disease or condition. It may be the treatment of an acute disease or condition in a patient having cancer, which does not mean necessarily that the cancer itself is treated. In a specific embodiment of the invention said critically ill patient does not suffer primarily from a chronic disease or condition. In a very specific embodiment of the invention said critically ill patient does not suffer from Addison disease.

Threshold levels can be obtained for instance from a Kaplan-Meier analysis, where the occurrence of a disease is correlated with the quartiles of the biomarker in the population. According to this analysis, subjects with biomarker levels above the 75th percentile have a significantly increased risk for getting the diseases according to the invention. This result is further supported by Cox regression analysis with full adjustment for classical risk factors: The highest quartile versus all other subjects is highly significantly associated with increased risk for getting a disease according to the invention.

Other preferred cut-off values are for instance the 90th, 95th or 99th percentile of a normal population. By using a higher percentile than the 75th percentile, one reduces the number of false positive subjects identified, but one might miss to identify subjects, who are at moderate, albeit still increased risk. Thus, one might adopt the cut-off value depending on whether it is considered more appropriate to identify most of the subjects at risk at the expense of also identifying “false positives”, or whether it is considered more appropriate to identify mainly the subjects at high risk at the expense of missing several subjects at moderate risk.

The above-mentioned threshold values might be different in other assays, if these have been calibrated differently from the assay system used in the present invention. Therefore, the above-mentioned threshold shall apply for such differently calibrated assays accordingly, taking into account the differences in calibration. One possibility of quantifying the difference in calibration is a method comparison analysis (correlation) of the assay in question (e.g., bio-ADM assay) with the respective biomarker assay used in the present invention by measuring the respective biomarker (e.g., bio-ADM) in samples using both methods. Another possibility is to determine with the assay in question, given this test has sufficient analytical sensitivity, the median biomarker level of a representative normal population, compare results with the median biomarker levels as described in the literature (e.g., Weber et al. 2017. JALM 2(2): 222-233) and recalculate the calibration based on the difference obtained by this comparison. With the calibration used in the present invention, samples from normal (healthy) subjects have been measured: median plasma bio-ADM (mature ADM-NH₂) was 13.7 pg/ml (inter quartile range [IQR] 9.6-18.7 pg/mL) (Weber et al. 2017. JALM 2(2): 222-233).

Throughout the specification the “antibodies”, or “antibody fragments” or “non-Ig scaffolds” in accordance with the invention are capable to bind ADM, and thus are directed against ADM, and thus can be referred to as “anti-ADM antibodies”, “anti-ADM antibody fragments”, or “anti-ADM non-Ig scaffolds”.

Mature ADM, bio-ADM and ADM-NH₂ is used synonymously throughout this application and is a molecule according to SEQ ID No.: 4.

In a specific embodiment of the diagnostic method, said binder exhibits a binding affinity to pro-Adrenomedullin or a fragment thereof (which is not ADM-NH₂ according to SEQ ID No.: 4) and ADM-NH₂ of at least 10⁷ M⁻¹, preferred 10⁸ M⁻¹, preferred affinity is greater than 10⁹ M⁻¹, most preferred greater than 10¹⁰ M⁻¹. A person skilled in the art knows that it may be considered to compensate lower affinity by applying a higher dose of compounds and this measure would not lead out-of-the-scope of the invention.

To determine the affinity of the antibodies to Adrenomedullin, the kinetics of binding of Adrenomedullin to immobilized antibody was determined by means of label-free surface plasmon resonance using a Biacore 2000 system (GE Healthcare Europe GmbH, Freiburg, Germany). Reversible immobilization of the antibodies was performed using an anti-mouse Fc antibody covalently coupled in high density to a CM5 sensor surface according to the manufacturer's instructions (mouse antibody capture kit; GE Healthcare), (Lorenz et al. 2011. Antimicrob Agents Chemother. 55 (1): 165-173).

In a specific embodiment of the method, an assay is used for determining the level ADM-NH₂ wherein such assay is a sandwich assay, preferably a fully automated assay.

In one embodiment of the invention, it may be a so-called POC-test (point-of-care) that is a test technology, which allows performing the test within less than 1 hour near the patient without the requirement of a fully automated assay system. One example for this technology is the immunochromatographic test technology, e.g., a microfluidic device.

In one embodiment of the diagnostic method such an assay is a sandwich immunoassay using any kind of detection technology including but not restricted to enzyme label, chemiluminescence label, electrochemiluminescence label, preferably a fully automated assay. In one embodiment of the diagnostic method such an assay is an enzyme labeled sandwich assay. Examples of automated or fully automated assay comprise assays that may be used for one of the following systems: Roche Elecsys®, Abbott Architect®, Siemens Centauer®, Brahms Kryptor®, Biomerieux Vidas®, Alere Triage®.

A variety of immunoassays are known and may be used for the assays and methods of the present invention, these include: radioimmunoassays (“RIA”), homogeneous enzyme-multiplied immunoassays (“EMIT”), enzyme linked immunoadsorbent assays (“ELISA”), apoenzyme reactivation immunoassay (“ARIS”), dipstick immunoassays and immuno-chromatography assays.

In a preferred embodiment said label is selected from the group comprising chemiluminescent label, enzyme label, fluorescence label, radioiodine label.

The assays can be homogenous or heterogeneous assays, competitive and non-competitive assays. In one embodiment, the assay is in the form of a sandwich assay, which is a non-competitive immunoassay, wherein the molecule to be detected and/or quantified is bound to a first antibody and to a second antibody. The first antibody may be bound to a solid phase, e.g., a bead, a surface of a well or other container, a chip or a strip, and the second antibody is an antibody which is labeled, e.g. with a dye, with a radioisotope, or a reactive or catalytically active moiety. The amount of labeled antibody bound to the analyte is then measured by an appropriate method. The general composition and procedures involved with “sandwich assays” are well-established and known to the skilled person (The Immunoassay Handbook, Ed. David Wild, Elsevier LTD, Oxford; 3rd ed. (May 2005), ISBN-13: 978-0080445267; Hultschig C et al., Curr Opin Chem Biol. 2006 February; 10(1):4-10. PMID: 16376134).

In another embodiment the assay comprises two capture molecules, preferably antibodies which are both present as dispersions in a liquid reaction mixture, wherein a first labelling component is attached to the first capture molecule, wherein said first labelling component is part of a labelling system based on fluorescence- or chemiluminescence-quenching or amplification, and a second labelling component of said marking system is attached to the second capture molecule, so that upon binding of both capture molecules to the analyte a measurable signal is generated that allows for the detection of the formed sandwich complexes in the solution comprising the sample.

In another embodiment, said labeling system comprises rare earth cryptates or rare earth chelates in combination with fluorescence dye or chemiluminescence dye, in particular a dye of the cyanine type.

In the context of the present invention, fluorescence based assays comprise the use of dyes, which may for instance be selected from the group comprising FAM (5- or 6-carboxyfluorescein), VIC, NED, Fluorescein, Fluoresceinisothiocyanate (FITC), IRD-700/800, Cyanine dyes, such as CY3, CY5, CY3.5, CY5.5, Cy7, Xanthen, 6-Carboxy-2′,4′,7′,4,7-hexachlorofluorescein (HEX), TET, 6-Carboxy-4′,5′-dichloro-2′,7′-dimethodyfluorescein (JOE), N,N,N′,N′-Tetramethyl-6-carboxyrhodamine (TAMRA), 6-Carboxy-X-rhodamine (ROX), 5-Carboxyrhodamine-6G (R6G5), 6-carboxyrhodamine-6G (RG6), Rhodamine, Rhodamine Green, Rhodamine Red, Rhodamine 110, BODIPY dyes, such as BODIPY TMR, Oregon Green, Coumarines such as Umbelliferone, Benzimides, such as Hoechst 33258; Phenanthridines, such as Texas Red, Yakima Yellow, Alexa Fluor, PET, Ethidiumbromide, Acridinium dyes, Carbazol dyes, Phenoxazine dyes, Porphyrine dyes, Polymethin dyes, and the like.

In the context of the present invention, chemiluminescence based assays comprise the use of dyes, based on the physical principles described for chemiluminescent materials in (Kirk-Othmer, Encyclopedia of chemical technology, 4th ed., executive editor, J. I. Kroschwitz; editor, M. Howe-Grant, John Wiley & Sons, 1993, vol. 15, p. 518-562, incorporated herein by reference, including citations on pages 551-562). Preferred chemiluminescent dyes are acridiniumesters.

As mentioned herein, an “assay” or “diagnostic assay” can be of any type applied in the field of diagnostics. Such an assay may be based on the binding of an analyte to be detected to one or more capture probes with a certain affinity. Concerning the interaction between capture molecules and target molecules or molecules of interest, the affinity constant is preferably greater than 10⁸ M⁻¹.

In a specific embodiment of the method, at least one of said two binders is labeled in order to be detected.

The present invention further relates to a kit for carrying out the method of the invention, comprising detection reagents for determining the level of ADM-NH₂, in a sample from a patient, and reference data, such as a reference and/or threshold level, corresponding to a level of ADM-NH₂ in said sample between 20 and 150 pg/mL, more preferred between 30 and 100 pg/mL, even more preferred between 40 and 80 pg/mL, most preferred 70 pg/mL, wherein said reference data is preferably stored on a computer readable medium and/or employed in the form of computer executable code configured for comparing the determined level of ADM-NH₂ to said reference data.

In one embodiment of the method described herein, the method additionally comprises comparing the determined level of ADM-NH₂ in critically patients to a reference and/or threshold level, wherein said comparing is carried out in a computer processor using computer executable code.

The methods of the present invention may in part be computer-implemented. For example, the step of comparing the detected level of a marker, e.g., ADM-NH₂, with a reference and/or threshold level can be performed in a computer system. For example, the determined values may be entered (either manually by a health professional or automatically from the device(s) in which the respective marker level(s) has/have been determined) into the computer-system. The computer-system can be directly at the point-of-care (e.g., primary care unit or ED) or it can be at a remote location connected via a computer network (e.g., via the internet, or specialized medical cloud-systems, optionally combinable with other IT-systems or platforms such as hospital information systems (HIS)). Alternatively, or in addition, the associated therapy guidance and/or therapy stratification will be displayed and/or printed for the user (typically a health professional such as a physician).

With the above context, the following consecutively numbered embodiments provide further specific aspects of the invention:
1. A method of selection of critically ill patients for treatment with corticosteroids, which comprises determining the level of ADM-NH₂ or fragment thereof in a sample of bodily fluid of said patient, comparing said level of ADM-NH₂ or fragment thereof to a pre-determined threshold or to a previously measured level of ADM-NH₂ or fragment thereof, and at a value above said threshold appointing a therapy with corticosteroids, and at a value below said threshold renouncing a therapy with corticosteroids.
2. A method for corticosteroid therapy guidance and/or stratification in critically ill patients according to embodiment 1, the method comprising
   a. Providing a sample of bodily fluid of said patient, and
   b. Determining the level of ADM-NH₂ or fragment thereof in said sample, and
   c. Comparing said level of ADM-NH₂ or fragment thereof to a pre-determined threshold or to a previously measured level of ADM-NH₂ or fragment thereof, wherein the level of ADM-NH₂ or fragments thereof is indicative of whether an initiation of a corticosteroid therapy is required.
3. A method for corticosteroid therapy guidance and/or stratification in critically ill patients according to embodiments 1 and 2, wherein said critically ill patients are patients suffering from a disease selected from the group of severe infection, sepsis, septic shock, acute respiratory syndrome (ARDS), community acquired pneumonia (CAP), meningitis (e.g., bacterial or viral meningitis), corona virus infection disease (e.g., COVID-19), cardiopulmonary bypass surgery (CPB) and cardiac arrest.
4. A method for corticosteroid therapy guidance and/or stratification in critically ill patients according to embodiments 1 to 3, wherein the level of ADM-NH₂ or fragment thereof is determined by contacting said sample of bodily fluid with a capture binder that binds specifically to pro-Adrenomedullin or fragment thereof.
5. A method for corticosteroid therapy guidance and/or stratification in critically ill patients according to embodiments 1 to 4, wherein said determination comprises the use of a capture-binder that binds specifically to ADM-NH₂ or fragment thereof wherein said capture-binder may be selected from the group of antibodies, antibody fragment or non-IgG scaffold.
6. A method for corticosteroid therapy guidance and/or stratification in critically ill patients according to embodiments 1 to 5, wherein the level of ADM is determined in a bodily fluid sample of said subject and wherein said determination comprises the use of a capture-binder that binds specifically to level of ADM, wherein said capture-binder is immobilized on a surface.
7. A method for corticosteroid therapy guidance and/or stratification in critically ill patients according to embodiments 1 to 6, wherein the level of ADM is determined by different methods, e.g., immunoassays, activity assays, mass spectrometric methods.
8. A method for corticosteroid therapy guidance and/or stratification in critically ill patients according to embodiments 1 to 7, wherein the assay sensitivity of said assay for the detection of ADM-NH₂ is able to quantify ADM-NH₂ of healthy subjects and is <70 pg/ml, preferably <40 pg/ml and more preferably <10 pg/ml.
9. A method for corticosteroid therapy guidance and/or stratification in critically ill patients according to embodiments 1 to 8, wherein said patient is treated with corticosteroids selected from the group consisting of glucocorticoids or mineralcorticoids.
10. A method for corticosteroid therapy guidance and/or stratification in critically ill patients according to embodiments 1 to 9, wherein said glucocorticoids may be selected from the group comprising cortisone, hydrocortisone, prednisone, prednisolone, methylprednisolone, dexamethasone or betamethasone.
11. A method for corticosteroid therapy guidance and/or stratification in critically ill patients according to embodiments 1 to 10, wherein said mineralcorticoids may be selected from the group comprising fludrocortisone.
12. A method for corticosteroid therapy guidance and/or stratification in critically ill patients according to embodiments 1 to 11, wherein said Corona Virus is selected from the group comprising Sars-COV-1, Sars-COV-2, MERS-COV, in particular Sars-COV-2.
13. A method for corticosteroid therapy guidance and/or stratification in critically ill patients according to embodiments 1 to 12, wherein the bodily is selected from the group comprising whole blood, serum and plasma.
14. A method for corticosteroid therapy guidance and/or stratification in critically ill patients according to embodiments 1 to 13, wherein the threshold level of ADM-NH₂ is between 20 and 150 pg/mL, more preferred between 30 and 100 pg/mL, even more preferred between 40 and 80 pg/mL, most preferred said threshold level is 70 pg/mL.
15. A method for corticosteroid therapy guidance and/or stratification in critically ill patients according to embodiments 1 to 14, wherein further biomarkers may be measured in addition to ADM-NH₂ or fragments thereof.
16. A method for corticosteroid therapy guidance and/or stratification in critically ill patients according to embodiments 1 to 15, wherein said further biomarkers are selected from the group comprising D-Dimer, procalcitonin (PCT), C-reactive protein (CRP), lactate, penKid, NT-proBNP, BNP, white blood cell count, lymphocyte count, neutrophil count, hemoglobin, platelet count, albumin, alanine transaminase, creatinine, blood urea, lactate dehydrogenase, creatinin kinase, cardiac troponin I, prothrombin time, serum ferritin, interleukin-6 (IL-6), IL-10, IL-2, IL-7, interferon gamma (IF-γ), tumor necrosis factor-α (TNF-α), granulocyte colony-stimulating factor (GCSF), IP-10, MCP-1, MIP-1α.

With the above context, the following consecutively numbered further embodiments provide further specific aspects of the invention:
1. A method of corticosteroid therapy guidance and/or corticosteroid therapy stratification of critically ill patients, the method comprising:
   providing a sample of bodily fluid of said patient, and
   determining the level of ADM-NH₂ or fragment thereof in said sample, and
   comparing said level of ADM-NH₂ or fragment thereof to a pre-determined threshold or to a previously measured level of ADM-NH₂ or fragment thereof,
wherein the level of ADM-NH₂ or fragments thereof is indicative of whether an initiation of corticosteroid therapy is required.
2. A method of corticosteroid therapy guidance and/or corticosteroid therapy stratification of critically ill patients according to embodiment 1, wherein a therapy with corticosteroids is appointed at a level of ADM-NH₂ or fragments thereof above said threshold, and wherein a therapy with corticosteroids is withheld at a level of ADM-NH₂ or fragments thereof below said threshold.
3. A method of corticosteroid therapy guidance and/or corticosteroid therapy stratification of critically ill patients according to embodiment 1, wherein said critically ill patients are patients suffering from a disease selected from the group of severe infection, sepsis, septic shock, acute respiratory syndrome (ARDS), community acquired pneumonia (CAP), meningitis (e.g., bacterial or viral meningitis), corona virus infection disease (e.g., COVID-19), cardiopulmonary bypass surgery (CPB) and cardiac arrest.
4. A method of corticosteroid therapy guidance and/or corticosteroid therapy stratification of critically ill patients according to embodiments 1 and 3, wherein the level of ADM-NH₂ or fragment thereof is determined by contacting said sample of bodily fluid with a capture binder that binds specifically to ADM-NH₂ or fragment thereof.
5. A method of corticosteroid therapy guidance and/or corticosteroid therapy stratification of critically ill patients according to embodiment 4, wherein said capture binder binds specifically to the C-terminal part of ADM-NH₂ (SEQ ID No. 9) and wherein said capture binder specifically needs the C-terminal amide of ADM-NH₂ for binding.
6. A method corticosteroid therapy guidance and/or corticosteroid therapy stratification of critically ill patients according to embodiments 4 and 5, wherein said capture-binder may be selected from the group of antibodies, antibody fragment or non-IgG scaffold.
7. A method of corticosteroid therapy guidance and/or corticosteroid therapy stratification of critically ill patients according to embodiments 4 to 6, wherein said capture-binder is immobilized on a surface.
8. A method of corticosteroid therapy guidance and/or corticosteroid therapy stratification of critically ill patients ill according to embodiments 1 to 7, wherein the level of ADM-NH₂ is determined by a method selected from the group comprising, an immunoassay, an activity assay, a mass spectrometric method.
9. A method of corticosteroid therapy guidance and/or corticosteroid therapy stratification of critically ill patients according to embodiment 8, wherein the assay sensitivity of said immunoassay for the detection of ADM-NH₂ is able to quantify ADM-NH₂ of healthy subjects and is <70 pg/ml, preferably <40 pg/ml and more preferably <10 pg/ml.
10. A method of corticosteroid therapy guidance and/or corticosteroid therapy stratification of critically ill patients according to embodiments 1 to 9, wherein said patient is treated with corticosteroids selected from the group consisting of glucocorticoids or mineralcorticoids.
11. A method of corticosteroid therapy guidance and/or corticosteroid therapy stratification of critically ill patients according to embodiment 10, wherein said glucocorticoids may be selected from the group comprising cortisone, hydrocortisone, prednisone, prednisolone, methylprednisolone, dexamethasone or betamethasone.
12. A method of corticosteroid therapy guidance and/or corticosteroid therapy stratification of critically ill patients according to embodiment 10, wherein said mineralcorticoids may be selected from the group comprising fludrocortisone.
13. A method of corticosteroid therapy guidance and/or corticosteroid therapy stratification of critically ill patients according to embodiment 3, wherein said Corona Virus is selected from the group comprising Sars-COV-1, Sars-COV-2, MERS-COV, in particular Sars-CoV-2.
14. A method of corticosteroid therapy guidance and/or corticosteroid therapy stratification of critically ill patients according to embodiments 1 to 13, wherein the bodily is selected from the group comprising whole blood, serum and plasma.
15. A method of corticosteroid therapy guidance and/or corticosteroid therapy stratification of critically ill patients according to embodiments 1 to 14, wherein the threshold level of ADM-NH₂ is between 20 and 150 pg/mL, more preferred between 30 and 100 pg/mL, even more preferred between 40 and 80 pg/mL, most preferred said threshold level is 70 pg/mL.
16. A method of corticosteroid therapy guidance and/or corticosteroid therapy stratification of critically ill patients according to embodiments 1 to 15, wherein further biomarkers may be measured in addition to ADM-NH₂ or fragments thereof.
17. A method of corticosteroid therapy guidance and/or corticosteroid therapy stratification of critically ill patients according to embodiments 1 to 16, wherein said further biomarkers are selected from the group comprising D-Dimer, procalcitonin (PCT), C-reactive protein (CRP), lactate, penKid, NT-proBNP, BNP, white blood cell count, lymphocyte count, neutrophil count, hemoglobin, platelet count, albumin, alanine transaminase, creatinine, blood urea, lactate dehydrogenase, creatinin kinase, cardiac troponin I, prothrombin time, serum ferritin, interleukin-6 (IL-6), IL-10, IL-2, IL-7, interferon gamma (IF-γ), tumor necrosis factor-α (TNF-α), granulocyte colony-stimulating factor (GCSF), IP-10, MCP-1, MIP-1a.
18. Corticosteroids for use in the treatment of critically ill patients, wherein said patient is characterized in that the level of ADM-NH₂ or fragment thereof in a sample of bodily fluid of said patient is at a value above a pre-determined threshold or above a previously measured level of ADM-NH₂ or fragment thereof, when said level of ADM-NH₂ or fragment thereof is compared to said pre-determined threshold or to a previously measured level of ADM-NH₂ or fragment thereof, wherein said critically ill patients are patients suffering from a disease selected from the group of severe infection, sepsis, septic shock, acute respiratory syndrome (ARDS), community acquired pneumonia (CAP), meningitis (e.g., bacterial or viral meningitis), corona virus infection disease (e.g., COVID-19), cardiopulmonary bypass surgery (CPB) and cardiac arrest.
19. Corticosteroids for use in the treatment of critically ill patients, wherein said patient is characterized in that the level of ADM-NH₂ or fragment thereof has been determined in a sample of bodily fluid of said patient, wherein said level of ADM-NH₂ or fragment thereof has been compared to a pre-determined threshold or to a previously measured level of ADM-NH₂ or fragment thereof, and at a value above said threshold a therapy with corticosteroids is appointed, wherein said critically ill patients are patients suffering from a disease selected from the group of severe infection, sepsis, septic shock, acute respiratory syndrome (ARDS), community acquired pneumonia (CAP), meningitis (e.g., bacterial or viral meningitis), corona virus infection disease (e.g., COVID-19), cardiopulmonary bypass surgery (CPB) and cardiac arrest.

### FIGURE DESCRIPTION

FIG. 1: shows a typical bio-ADM dose/signal curve and a bio-ADM dose signal curve in the presence of 100 μg/mL antibody NT-H.

FIG. 2: Percentage of patients developing septic shock (light gray) by bio-ADM>/<70 pg/mL and treatment arm (PP population) (p=0.033).

FIG. 3: 90-day mortality by bio-ADM>/<70 pg/mL and treatment arm (PP population).

FIG. 4: Length of hospital stay by bio-ADM>/<70 pg/mL and treatment arm (PP population).

### EXAMPLES

### Example 1—Generation of Antibodies and Determination of their Affinity Constants

We developed mouse monoclonal antibodies binding to the N-terminal (NT-ADM), midregional (MR-ADM) and C-terminal (CT-ADM) part of bio-ADM and their affinity constants were determined (Table 2).

### Peptides for Immunization

Peptides were supplied by JPT Peptide Technologies GmbH (Berlin, Germany). Peptides were coupled to BSA using the Sulfo-SMCC crosslinking method. The crosslinking procedure was performed according the manufacturer's instructions (Thermo Fisher/Pierce).

### Generation of Murine Antibodies

A Balb/c mouse was immunized with 100 μg Peptide-BSA-Conjugate at day 0 and 14 (emulsified in 100 μl complete Freund's adjuvant) and 50 μg at day 21 and 28 (in 100 μl incomplete Freund's adjuvant). Three days before the fusion experiment was performed, the animal received 50 μg of the conjugate dissolved in 100 μl saline, given as one intraperitoneal and one intra venous injection.

Splenocytes from the immunized mouse and cells of the myeloma cell line SP2/0 were fused with 1 ml 50% polyethylene glycol for 30 s at 37° ° C. After washing, the cells were seeded in 96-well cell culture plates. Hybrid clones were selected by growing in HAT medium (RPMI 1640 culture medium supplemented with 20% fetal calf serum and HAT-Supplement). After two weeks the HAT medium is replaced with HT medium for three passages followed by returning to the normal cell culture medium.

The cell culture supernatants were primary screened for antigen specific IgG antibodies three weeks after fusion. The positive tested microcultures were transferred into 24-well plates for propagation. After retesting the selected cultures were cloned and recloned using the limiting-dilution technique and the isotypes were determined (Lane, 1985. J. Immunol. Meth. 81: 223-228; Ziegler et al. 1996. Horm. Metab. Res. 28: 11-15).

TABLE 2

| | ADM | | Affinity constants |
|---|---|---|---|
| Antigen/Immunogen | Region | Designation | Kd (M⁻¹) |
| YRQSMNNFQGLRSFGC (SEQ ID No. 7) | 1-16 | NT-ADM | 1.6 × 109 |
| CTVQKLAHQIYQ (SEQ ID No. 8) | 21-32 | MR-ADM | 2 × 109 |
| CAPRSKISPQGY-NH₂ (SEQ ID No. 9) | C-42-52 | CT-ADM | 1.1 × 109 |

### Monoclonal Antibody Production

Antibodies were produced via standard antibody production methods (Marx et al, 1997. Monoclonal Antibody Production, ATLA 25, 121) and purified via Protein A. The antibody purities were >95% based on SDS gel electrophoresis analysis.

### Affinity Constants

To determine the affinity of the antibodies to Adrenomedullin, the kinetics of binding of Adrenomedullin to immobilized antibody was determined by means of label-free surface plasmon resonance using a Biacore 2000 system (GE Healthcare Europe GmbH, Freiburg, Germany). Reversible immobilization of the antibodies was performed using an anti-mouse Fc antibody covalently coupled in high density to a CM5 sensor surface according to the manufacturer's instructions (mouse antibody capture kit; GE Healthcare).

### Labelling Procedure (Tracer)

100 μg (100 μl) of antibody (1 mg/ml in PBS, pH 7.4) was mixed with 10 μl Akridinium NHS-ester (1 mg/ml in acetonitrile, InVent GmbH, Germany) (EP 0 353 971) and incubated for 20 min at room temperature. Labelled CT-H was purified by Gel-filtration HPLC on Bio-Sil® SEC 400-5 (Bio-Rad Laboratories, Inc., USA). The purified labeled antibody was diluted in (300 mmol/L potassiumphosphate, 100 mmol/L NaCl, 10 mmol/L Na-EDTA, 5 g/L Bovine Serum Albumin, pH 7.0). The final concentration was approx. 800.000 relative light units (RLU) of labelled compound (approx. 20 ng labeled antibody) per 200 μL. Akridiniumester chemiluminescence was measured by using an AutoLumat LB 953 (Berthold Technologies GmbH & Co. KG).

### Solid Phase

Polystyrene tubes (Greiner Bio-One International AG, Austria) were coated (18 h at room temperature) with antibody (1.5 μg antibody/0.3 mL 100 mmol/L NaCl, 50 mmol/L TRIS/HCl, pH 7.8). After blocking with 5% bovine serum albumin, the tubes were washed with PBS, pH 7.4 and vacuum dried.

### Calibrators

Synthetic human ADM (hADM) (Bachem, Switzerland) was linearily diluted using 50 mM Tris/HCl, 250 mM NaCl, 0.2% Triton X-100, 0.5% BSA, 20 tabs/L Protease Complete Protease Inhibitor Cocktail Tablets (Roche AG); pH 7.8. Calibrators were stored at −20° C. before use.

### Example 2—Determination of the Antibody Combination that Yields High Signal/Noise Ratios

### ADM Immunoassay

50 μl of sample (or calibrator) was pipetted into coated tubes, after adding labelled second antibody (200 μl), the tubes were incubated for 2 h at room temperature. Unbound tracer was removed by washing 5 times (each 1 ml) with washing solution (20 mM PBS, pH 7.4, 0.1% Triton X-100). Tube-bound chemiluminescence was measured by using the LB 953 (Berthold Technologies Gmbh & Co. KG).

All antibodies were used in a sandwich immunoassay as coated tube and labelled antibody and combined in the following variations (see Table 3). Incubation was performed as described under hADM-Immunoassay. Results are given in ratio of specific signal (at 10 ng/ml ADM)/background (sample without ADM) signal.

TABLE 3

| | NT-ADM | MR-ADM | CT-ADM |
|---|---|---|---|
| Signal/noise ratio | tracer | tracer | tracer |
| NT-ADM | / | 195 | 241 |
| MR-ADM | 204 | / | 904 |
| CT-ADM | 260 | 871 | / |

Surprisingly, we found the combination of MR-ADM and CT-ADM as combination for highest signal/noise ratio.

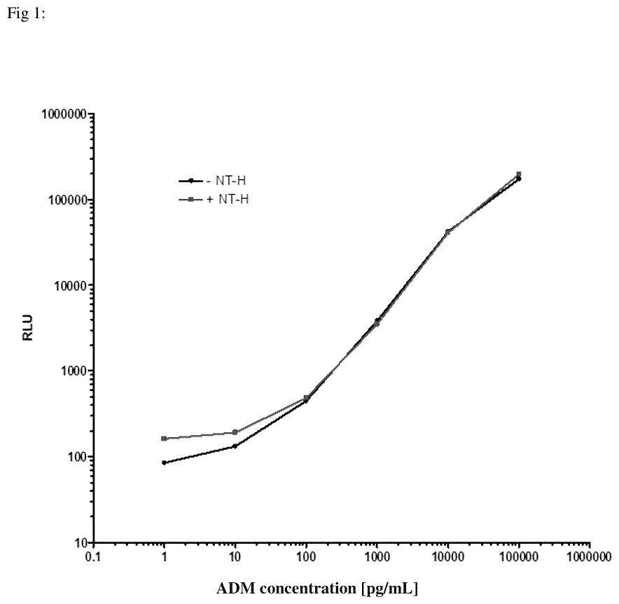

Subsequently, we used this antibody-combination for further investigations to measure bio-ADM. We used anti-MR-ADM as solid phase antibody and anti-CT-ADM as labelled antibody. A typical dose/signal curve is shown in FIG. 1. The analytical sensitivity (average of 10 runs, ADM-free sample+2SD) of the assay was 2 pg ADM/ml.

### Example 3—Stability of Human Adrenomedullin

Human ADM was diluted in human Citrate plasma (n=5, final concentration 10 ng ADM/ml) and incubated at 24° C. At selected time points, aliquots were frozen at −20° C. Immediately after thawing the samples hADM was quantified by using the hADM immunoassay described above.

Table 4 shows the stability of hADM in human plasma at 24° C.

| Time | Average ADM | Relative loss of | Loss of immune |
|---|---|---|---|
| (h) | recovery (N = 5) | immune reactivity | reactivity %/h |
| 0 | 100 | / | / |
| 2 | 99.2 | 0.8 | 0.4 |
| 4 | 96.4 | 3.6 | 0.8 |
| 8 | 88.2 | 11.8 | 1.5 |
| | | | Average: 0.9%/h |

Surprisingly, using the antibody-combinations MR-ADM and CT-ADM in a sandwich immune assay, the pre-analytical stability of the analyte is high (only 0.9%/h average loss of immune reactivity). In contrast, using other assay methods, a plasma half-life of only 22 min was reported (Hinson et al. 2000 Endocrine Reviews 21(2): 138-167). Since the time from taking sample to analysis in hospital routine is less than 2 h, the used ADM detection method is suitable for routine diagnosis. It is remarkable, that any non-routine additives to samples (like aprotinin, (Ohta et al. 1999. Clin Chem 45 (2): 244-251)) are not needed to reach acceptable ADM-immune reactivity stabilities.

### Example 4—Reproducibility of Calibrator-Preparations

We found a high variation of results, preparing calibrators for ADM assays (average CV 8.5%, see Table 4). This may be due to high adsorption of hADM to plastic and glass surfaces (Lewis et al. 1998. Clinical Chemistry 44 (3): 571-577). This effect was only slightly reduced by adding detergents (up to 1% Triton X 100 or 1% Tween 20), protein (up to 5% BSA) and high ionic strength (up to 1M NaCl) or combinations thereof. Surprisingly, if a surplus of anti-ADM antibody (10 μg/ml) is added to the calibrator dilution buffer, the recovery and reproducibility of ADM assay calibrator-preparations was substantially improved to <1% of inter preparation CV (Table 5).

Fortunately, the presence of N-terminal antibodies did not affect the bio-ADM-signal generated by the combination of MR- and C-terminal antibodies (FIG. 1).

TABLE 5

| | | In the | | | | | |
|---|---|---|---|---|---|---|---|
| | | presence of | | | | | |
| | | NT-ADM | | Inter | | | Inter |
| | | antibody | | preparation | Without | | preparation |
| | | (10 μg/ml) | | CV (%) | antibody | | CV (%) |
| calibrator | | | | | | | |
| 100 | ng/ml | 3453 | s/n-r | 0.9 | 2842 | s/n-r | 2.8 |
| 10 | ng/ml | 1946 | s/n-r | 0.8 | 1050 | s/n-r | 7.9 |
| 1 | ng/ml | 179 | s/n-r | 1.1 | 77 | s/n-r | 14.8 |

| | |
|---|---|
| Average: 0.93 | Average: 8.5 |

### Inter-Preparation Variation of Calibrators

ADM assay calibrators were prepared as described above with and without 10 μg/ml of NT-ADM-antibody. Coefficients of variation are given from 5 independent preparation runs. The calibrators were measured using the ADM assay described above (s/n−r=signal to noise ratio). For all following studies, we used an ADM assay, based on calibrators, prepared in the presence of 10 μg/ml of NT-ADM antibody and 10 μg/ml of NT-ADM antibody as supplement in the tracer buffer.

### Example 5

### Sensitivity

The goal of assay sensitivity was to completely cover the ADM concentration of healthy subjects.

### Bio-ADM Concentration in Healthy Subjects

Healthy subjects (n=100, average age 56 years) were measured using the bio-ADM assay. The median value was 24.7 pg/ml, the lowest value 11 pg/ml and the 99^{th} percentile 43 pg/ml. Since the assay sensitivity was 2 pg/ml, 100% of all healthy subjects were detectable using the described bio-ADM assay.

### Example 6—HYPRESS Study

### Study Details

The HYPRESS study is a double-blind, randomized clinical trial conducted from Jan. 13, 2009, to Aug. 27, 2013, with a follow-up of 180 days until Feb. 23, 2014 (Keh et al. 2016. JAMA 316(17): 1775-1785). The trial was performed in 34 intermediate or intensive care units of university and community hospitals in Germany, and it included 380 adult patients with severe sepsis who were not in septic shock.

Patients were screened in intermediate care units or intensive care units (ICUs) of university and community hospitals for eligibility, and written informed consent was obtained from patients, patient-authorized representatives, or legal representatives. Patients were enrolled if they met all inclusion criteria: (1) provided informed consent; (2) had evidence of infection; (3) had evidence of a systemic response to infection, defined as at least 2 systemic inflammatory response syndrome criteria 12; and (4) had evidence of organ dysfunction present for not longer than 48 hours. The main exclusion criterion was septic shock. Other exclusion criteria were being younger than 18 years, having known hypersensitivity to hydrocortisone or mannitol (placebo), or having a history of glucocorticoid medication with indication for continuation of therapy or other indications for treatment with glucocorticoids. Patients were not excluded for using etomidate within 72 hours before enrolment, using a short course of glucocorticoids within 72 hours before enrolment, or using topical or inhaled glucocorticoids.

Septic shock was defined as sepsis-induced hypotension despite adequate volume status for longer than 4 hours (ie, mean arterial pressure <65 mm Hg, systolic arterial pressure <90 mm Hg, or the use of vasopressors to keep mean arterial pressure ≥65 mm Hg or systolic arterial pressure ≥90 mm Hg). Patients who had a transient need for vasopressors during initial resuscitation but were not hypotensive and did not use vasopressors for at least 2 hours were eligible for enrolment when septic shock was not present at the time of randomization. Adequate volume status was defined as a central venous pressure of 8 mm Hg or greater (≥12 mm Hg in ventilated patients) and a central venous oxygen saturation greater than 70%. For fluid replacement, patients were to receive at least 500 to 1000 mL of crystalloids or 300 to 500 mL of colloids over 30 minutes. The use of hydroxyethyl starch preparations was discouraged owing to possible harmful effects on kidney function. Use of vasopressors was defined as therapy with dopamine at a dosage of at least 5 μg/kg/min or with any dose of epinephrine, norepinephrine, vasopressin, or other vasopressors.

The study medication (hydrocortisone and placebo) was produced and released by BAG Health Care GmbH. The medication was delivered in boxes, each containing 17 brown glass vials for 1 patient. Each vial contained 100 mg of lyophilized hydrocortisone hydrogen succinate or the same amount of lyophilized mannitol as placebo, which was indistinguishable from hydrocortisone. The medication was administered as an intravenous bolus of 50 mg, followed by a 24-hour continuous infusion of 200 mg on 5 days, 100 mg on days 6 and 7, 50 mg on days 8 and 9, and 25 mg on days 10 and 11.

For the present analysis, a total of n=110 cases and controls from the intention-to-treat data set of the SepNet study HYPRESS study (ClinicalTrials.gov number: NCT00670254) were selected, of which n=97 were treated according to protocol and selected for the analysis. Patients with and without septic shock during their ICU stay were selected at a 1:1 ratio. Control patients were matched according to demographics, organ dysfunction, concomitant medication and other outcomes.

The primary end point for the case-control study was the occurrence of septic shock within 28 days, or discharge from the ICU. The goal was to identify potential interaction between the levels of bio-ADM (</>70 pg/mL) and treatment arm (hydrocortisone vs. placebo).

### Results

Of the n=97 patients from the PP population, n=47 (49%) were treated with hydrocortisone, n=41 (42%) had elevated bio-ADM on enrolment (>70 pg/mL) and n=25 (26%) died within 90 days. Median bio-ADM in patients treated with hydrocortisone was 67 pg/mL, and 56 pg/mL in placebo (p=0.279). Bio-ADM predicted septic shock, independent of treatment arm: 61% (n=25/41) of patients with bio-ADM>70 pg/mL developed septic shock, while only 36% (n=20/56) did so if bio-ADM<70 pg/mL (p=0.024).

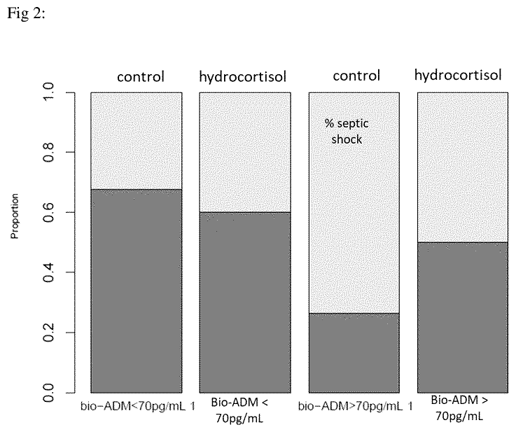

In patients with bio-ADM<70 pg/mL, 40% developed shock if treated with hydrocortisone, compared to 32.3% in the placebo group. In patients with bio-ADM>70 pg/mL, 50% developed shock if treated with hydrocortisone, compared to 73.7% in the placebo group (p-value comparing all 4 groups=0.033; p-value for interaction=0.119, FIG. 2).

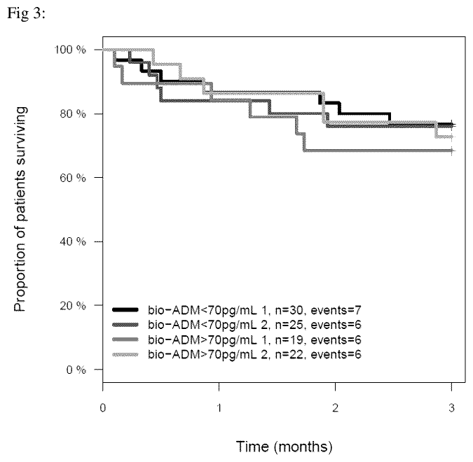

In patients with bio-ADM<70 pg/mL, 76.0% survived the 90-day follow up if treated with hydrocortisone, compared to 76.7% in the placebo group. In patients with bio-ADM>70 pg/mL, 72.7% survived the 90-day follow up if treated with hydrocortisone, compared to 68.4% in the placebo group (p-value for interaction=n.s., FIG. 3).

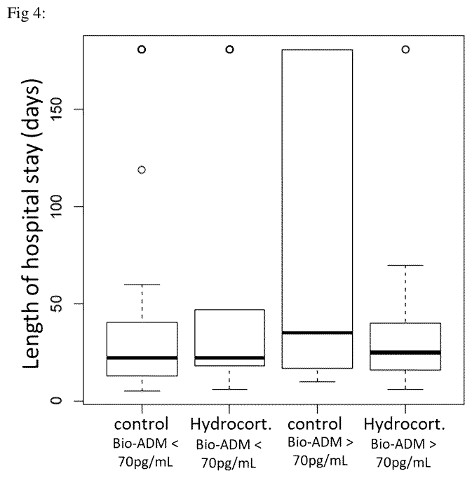

Supporting this finding, patients with bio-ADM>70 pg/mL in the control arm remained in hospital longer and needed more additional treatment. In patients with bio-ADM>70 pg/mL, median length of stay was 25 days if treated with hydrocortisone, compared to 35 days in the placebo group. In patients with bio-ADM<70 pg/mL, median length of stay was 22 days if treated with hydrocortisone, compared to 22 days in the placebo group (p-value comparing all 4 groups=0.597, FIG. 4).

In patients with bio-ADM>70 pg/mL, only 27.3% needed a surgical intervention treated with hydrocortisone, compared to 57.9% in the placebo group. In patients with bio-ADM<70 pg/mL, 48.0% needed a surgical intervention if treated with hydrocortisone, compared to 61.3% in the placebo group (p-value comparing all 4 groups=0.085).

In patients with bio-ADM>70 pg/mL, only 4.5% needed additional treatment with Propofol if treated with hydrocortisone, compared to 31.6% in the placebo group. In patients with bio-ADM<70 pg/mL, 16.0% needed additional treatment with Propofol if treated with hydrocortisone, compared to 12.9% in the placebo group (p-value comparing all 4 groups=0.114).

In patients with bio-ADM>70 pg/mL, only 13.6% needed additional treatment with Benzodiazepine if treated with hydrocortisone, compared to 31.6% in the placebo group. In patients with bio-AD<70 pg/mL, 12.0% needed additional treatment with Benzodiazepine if treated with hydrocortisone, compared to 12.9% in the placebo group (p-value comparing all 4 groups=0.282).

In patients with bio-ADM>70 pg/mL, only 31.8% needed additional treatment with opioids if treated with hydrocortisone, compared to 57.9% in the placebo group. In patients with bio-ADM<70 pg/mL, 40.0% needed additional treatment with opioids if treated with hydrocortisone, compared to 41.9% in the placebo group (p-value comparing all 4 groups=0.401).

SEQUENCES

SEQ ID No.: 1 (preproADM: 185 amino acids)
MKLVSVALMYLGSLAFLGADTARLDVASEFRKKWNKWALSRGKRELRMSSSYPTG
LADVKAGPAQTLIRPQDMKGASRSPEDSSPDAARIRVKRYRQSMNNFQGLRSFGCRF
GTCTVQKLAHQIYQFTDKDKDNVAPRSKISPQGYGRRRRRSLPEAGPGRTLVSSKPQ
AHGAPAPPSGSAPHFL
SEQ ID No.: 2 (Proadrenomedullin N-20 terminal peptide, PAMP: amino acids 22-41 of
preproADM)
ARLDVASEF RKKWNKWALS R
SEQ ID No.: 3 (Midregional proAdrenomedullin, MR-proADM: amino acids 45 - 92 of
preproADM)
ELRMSS SYPTGLADVK AGPAQTLIRP QDMKGASRSP EDSSPDAARI RV
SEQ ID No .: 4 (mature human Adrenomedullin (mature ADM); amidated ADM; bio-ADM):
amino acids 1-52 or amino acids 95-146 of pro-ADM
YRQSMNNFQGLRSFGCRFGTCTVQKLAHQIYQFTDKDKDNVAPRSKISPQGY-
CONH₂
SEQ ID No.: 5 (Adrenomedullin 1-52-Gly (ADM 1-52-Gly): amino acids 95-147 of
preproADM)
YRQSMN NFQGLRSFGC RFGTCTVQKL AHQIYQFTDK DKDNVAPRSK ISPQGYG
SEQ ID No.: 6 (C-terminal proAdrenomedullin, CT-proADM: amino acids 148-185 of
preproADM)
RRR RRSLPEAGPG RTLVSSKPQA HGAPAPPSGS APHFL
SEQ ID No.: 7 (human ADM 1-21)
YRQSMNNFQGLRSFGCRFGTC
SEQ ID No.: 8 (human ADM 21-32)
CTVQKLAHQIYQ
SEQ ID No.: 9 (human ADM C-42-52)
CAPRSKISPQGY-CONH₂

1. A method comprising:
treating a subject with corticosteroids,
wherein the level of ADM-NH₂ or fragment thereof in a sample of bodily fluid of a patient is more than 20 pg/ml.

2. (canceled)

3. The method of claim 1, wherein said patients are patients suffering from a disease selected from the group consisting of severe infection, sepsis, septic shock, acute respiratory syndrome (ARDS), community acquired pneumonia (CAP), meningitis, corona virus infection disease, cardiopulmonary bypass surgery (CPB) and cardiac arrest.

4. (canceled)

5. (canceled)

6. (canceled)

7. A The method of claim 1, wherein said patient is treated with corticosteroids selected from the group consisting of glucocorticoids or mineralcorticoids.

8. The method of claim 7, wherein said glucocorticoids are selected from the group consisting of cortisone, hydrocortisone, prednisone, prednisolone, methylprednisolone, dexamethasone and betamethasone.

9. The method of claim 7, wherein said mineralcorticoids is fludrocortisone.

10. The method of claim 3, wherein said Corona Virus is selected from the group consisting of Sars-COV-1, Sars-COV-2, MERS-COV, in particular Sars-COV-2.

11. The method of claim 1, wherein the bodily fluid is selected from the group consisting of whole blood, serum, and plasma.

12. The method of claim 1, wherein the level of ADM-NH₂ or fragment thereof in said sample is 70 pg/mL.

13. The method of claim 1, additionally comprising measuring the level of at least one additional biomarker in addition to ADM-NH₂ or fragments thereof.

14. The method of claim 13, wherein said at least one additional biomarker is selected from the group consisting of D-Dimer, procalcitonin (PCT), C-reactive protein (CRP), lactate, DPP3, penKid, NT-proBNP, BNP, white blood cell count, lymphocyte count, neutrophil count, hemoglobin, platelet count, albumin, alanine transaminase, creatinine, blood urea, lactate dehydrogenase, creatinin kinase, cardiac troponin I, prothrombin time, serum ferritin, interleukin-6 (IL-6), IL-10, IL-2, IL-7, interferon gamma (IF-γ), tumor necrosis factor-α (TNF-α), granulocyte colony-stimulating factor (GCSF), IP-10, MCP-1, and MIP-1α.

15. (canceled)

16. (canceled)

17. A sample comprising:
bodily fluid from a subject and a capture binder, wherein said capture binder is the product a process comprising:
fusing spenocytes from a mouse immunized with a peptide-BSA-conjugate and cells of a myeloma cell line
screening for capture binders which bind to ADM-NH₂ or fragment thereof,
wherein said subject has been diagnosed as critically ill, and
wherein the level of ADM-NH₂ or fragment thereof in said sample is more than 20 pg/ml.

18. The method of claim 1, wherein the level of ADM-NH₂ or fragment thereof in said sample is 40 pg/mL.


SEQUENCE LISTING (9 sequences)

SEQ ID NO: 1 - protein, 185 aa, Human
MKLVSVALMYLGSLAFLGADTARLDVASEFRKKWNKWALSRGKRELRMSSSYPTGLADVKAGPAQTLIRPQDMKGASRSPEDSSPDAARIRVKRYRQSMNNFQGLRSFGCRFGTCTVQKLAHQIYQFTDKDKDNVAPRSKISPQGYGRRRRRSLPEAGPGRTLVSSKPQAHGAPAPPSGSAPHFL

SEQ ID NO: 2 - protein, 20 aa, Human
ARLDVASEFRKKWNKWALSR

SEQ ID NO: 3 - protein, 48 aa, Human
ELRMSSSYPTGLADVKAGPAQTLIRPQDMKGASRSPEDSSPDAARIRV

SEQ ID NO: 4 - protein, 52 aa, Human
YRQSMNNFQGLRSFGCRFGTCTVQKLAHQIYQFTDKDKDNVAPRSKISPQGY

SEQ ID NO: 5 - protein, 53 aa, Human
YRQSMNNFQGLRSFGCRFGTCTVQKLAHQIYQFTDKDKDNVAPRSKISPQGYG

SEQ ID NO: 6 - protein, 38 aa, Human
RRRRRSLPEAGPGRTLVSSKPQAHGAPAPPSGSAPHFL

SEQ ID NO: 7 - protein, 21 aa, Human
YRQSMNNFQGLRSFGCRFGTC

SEQ ID NO: 8 - protein, 12 aa, Human
CTVQKLAHQIYQ

SEQ ID NO: 9 - protein, 12 aa, Human
CAPRSKISPQGY